(19) United States
(12) Patent Application Publication
(10) Pub. No.: US 2014/0179807 A1
(43) Pub. Date: Jun. 26, 2014

(54) MODULE-LEVEL ANALYSIS OF PERIPHERAL BLOOD LEUKOCYTE TRANSCRIPTIONAL PROFILES

(71) Applicants: Damien Chaussabel, Richardson, TX (US); Jacques Banchereau, Dallas, TX (US)

(72) Inventors: Damien Chaussabel, Richardson, TX (US); Jacques Banchereau, Dallas, TX (US)

(73) Assignee: Baylor Research Institute, Dallas, TX (US)

(21) Appl. No.: 14/056,776

(22) Filed: Oct. 17, 2013

Related U.S. Application Data
(62) Division of application No. 11/608,815, filed on Dec. 9, 2006, now abandoned.
(60) Provisional application No. 60/748,884, filed on Dec. 9, 2005.

(51) Int. Cl. G06F 19/24; C12Q 1/68

(52) U.S. Cl. CPC G06F 19/24; C12Q 1/6883
USPC 514/789; 435/6.11; 506/9; 435/6.12; 506/8; 435/7.92; 435/7.1

(57) ABSTRACT

The present invention includes an apparatus, system and method for the development and use of transcriptional modules by obtaining individual gene expression levels from cells obtained from one or more patients with a disease or condition; recording the expression value for each gene in a table that is divided into clusters; iteratively selecting gene expression values for one or more transcriptional modules by: selecting for the module the genes from each cluster that match in every disease or condition; removing the selected genes from the analysis; and repeating the process of gene expression value selection for genes that cluster in a sub-fraction of the diseases or conditions; and iteratively repeating the generation of modules.

### CROSS-REFERENCE TO RELATED APPLICATIONS

This application is a divisional of co-pending U.S. patent application Ser. No. 11/608,815 filed Dec. 9, 2006, which claims priority to U.S. Provisional Application Ser. No. 60/748,884 filed Dec. 9, 2005. The contents of each of these applications is specifically incorporated herein by reference in its entirety.

### STATEMENT OF FEDERALLY FUNDED RESEARCH

This invention was made with U.S. Government support under Contract Nos. U19 AI057234-02, P01 CA084512 and R01 CA078846 awarded by DARPA and the NIH. The government has certain rights in this invention. Without limiting the scope of the invention, its background is described in connection with gene mining.

### TECHNICAL FIELD OF THE INVENTION

The present invention relates in general to the transcriptional profiling of cells, and more particularly, to the diagnosis and prognosis of disease from the transcriptional expression profiles of leukocytes.

### LENGTHY TABLE

The present application includes a TABLE filed electronically via EFS-Web that includes the following tables in Landscape.

| | File Name | Sizes in Bytes | Dates of Creation |
|---|---|---|---|
| 1. | Modules - Round 1 | 145,993 | Dec. 9, 2006 |
| 2. | Modules - Round 2 | 223,210 | Dec. 9, 2006 |
| 3. | Modules - Round 3 | 310,185 | Dec. 9, 2006 |

A copy of the table is available in electronic form from the USPTO web site. An electronic copy of the table will also be available from the USPTO upon request and payment of the fee set forth in 37 CFR 1.19(b)(3).

LENGTHY TABLES
The patent application contains a lengthy table section. A copy of the table is available in electronic form from the USPTO web site (http://seqdata.uspto.gov/?pageRequest=docDetail&DocID=US20140179807A1). An electronic copy of the table will also be available from the USPTO upon request and payment of the fee set forth in 37 CFR 1.19(b)(3).

### BACKGROUND OF THE INVENTION

The widespread utilization of gene expression microarrays holds great promise for biomedical research. This technology has led to the establishment of prognostic signatures in cancer patients¹⁻⁴ and the identification of genes or pathways involved in pathogenesis (for instance, the discovery of the role of interleukin-1 (IL-1) in the pathogenesis of systemic onset juvenile idiopathic arthritis)⁵. However, despite these significant advances, gene expression microarray technology has not lived up to the excitement surrounding its inception, and results derived from the use of microarray platforms have recently been the object of sharp criticisms⁶. Among the chief concerns is the fact that microarray data are particularly prone to noise and could, when over-interpreted, lead to the generation of spurious results⁷. Skepticism also stems from notoriously poor reproducibility of microarray data obtained by different laboratories and across platforms⁸⁻¹². Finally, the limited ability to interpret experimental results in a genome-wide context constitutes another bottleneck in microarray research¹³.

### SUMMARY OF THE INVENTION

Genomic research is facing significant challenges with the analysis of transcriptional data that are notoriously noisy, difficult to interpret and do not compare well across laboratories and platforms. The present inventors have developed an analytical strategy emphasizing the selection of biologically relevant genes at an early stage of the analysis, which are consolidated into analytical modules that overcome the inconsistencies among microarray platforms. The transcriptional modules developed may be used for the analysis of large gene expression datasets. The results derived from this analysis are easily interpretable and particularly robust, as demonstrated by the high degree of reproducibility observed across commercial microarray platforms.

Applications for this analytical process are illustrated through the mining of a large set of PBMC transcriptional profiles. Twenty-eight transcriptional modules regrouping 4742 genes were identified. Using the present invention is it possible to demonstrate that diseases are uniquely characterized by combinations of transcriptional changes in, e.g., blood leukocytes, measured at the modular level. Indeed, module-level changes in blood leukocytes transcriptional levels constitute the molecular fingerprint of a disease or sample.

This invention has a broad range of applications. It can be used to characterize modular transcriptional components of any biological system (e.g., peripheral blood mononuclear cells (PBMCs), blood cells, fecal cells, peritoneal cells, solid organ biopsies, resected tumors, primary cells, cells lines, cell clones, etc.). Modular PBMC transcriptional data generated through this approach can be used for molecular diagnostic, prognostic, assessment of disease severity, response to drug treatment, drug toxicity, etc. Other data processed using this approach can be employed for instance in mechanistic studies, or screening of drug compounds. In fact, the data analysis strategy and mining algorithm can be implemented in generic gene expression data analysis software and may even be used to discover, develop and test new, disease- or condition-specific modules. The present invention may also be used in conjunction with pharmacogenomics, molecular diagnostic, bioinformatics and the like, wherein in-depth expression data may be used to improve the results (e.g., by improving or sub-selecting from within the sample population) that mat be obtained during clinical trails.

More particularly, the present invention includes arrays, apparatuses, systems and method for diagnosing a disease or condition by obtaining the transcriptome of a patient; analyzing the transcriptome based on one or more transcriptional modules that are indicative of a disease or condition; and determining the patient's disease or condition based on the presence, absence or level of expression of genes within the transcriptome in the one or more transcriptional modules. The transcriptional modules may be obtained by: iteratively selecting gene expression values for one or more transcriptional modules by: selecting for the module the genes from each cluster that match in every disease or condition; removing the selected genes from the analysis; and repeating the process of gene expression value selection for genes that cluster in a sub-fraction of the diseases or conditions; and iteratively repeating the generation of modules for each clusters until all gene clusters are exhausted.

Examples of clusters selected for use with the present invention include, but are not limited to, expression value clusters, keyword clusters, metabolic clusters, disease clusters, infection clusters, transplantation clusters, signaling clusters, transcriptional clusters, replication clusters, cell-cycle clusters, siRNA clusters, miRNA clusters, mitochondrial clusters, T cell clusters, B cell clusters, cytokine clusters, lymphokine clusters, heat shock clusters and combinations thereof. Examples of diseases or conditions for analysis using the present invention include, e.g., autoimmune disease, a viral infection a bacterial infection, cancer and transplant rejection. More particularly, diseases for analysis may be selected from one or more of the following conditions: systemic juvenile idiopathic arthritis, systemic lupus erythematosus, type I diabetes, liver transplant recipients, melanoma patients, and patients bacterial infections such as Escherichia coli, Staphylococcus aureus, viral infections such as influenza A, and combinations thereof. Specific array may even be made that detect specific diseases or conditions associated with a bioterror agent.

Cells that may be analyzed using the present invention, include, e.g., peripheral blood mononuclear cells (PBMCs), blood cells, fetal cells, peritoneal cells, solid organ biopsies, resected tumors, primary cells, cells lines, cell clones and combinations thereof. The cells may be single cells, a collection of cells, tissue, cell culture, cells in bodily fluid, e.g., blood. Cells may be obtained from a tissue biopsy, one or more sorted cell populations, cell culture, cell clones, transformed cells, biopies or a single cell. The types of cells may be, e.g., brain, liver, heart, kidney, lung, spleen, retina, bone, neural, lymph node, endocrine gland, reproductive organ, blood, nerve, vascular tissue, and olfactory epithelium cells. After cells are isolated, these mRNA from these cells is obtained and individual gene expression level analysis is performed using, e.g., a probe array, PCR, quantitative PCR, bead-based assays and combinations thereof. The individual gene expression level analysis may even be performed using hybridization of nucleic acids on a solid support using cDNA made from mRNA collected from the cells as a template for reverse transcriptase.

In another embodiment, the present invention includes a method for identifying transcriptional modules by obtaining individual gene expression levels from cells obtained from one or more patients with a disease or condition; recording the expression value for each gene in a table that is divided into clusters; iteratively selecting gene expression values for one or more transcriptional modules by: selecting for the module the genes from each cluster that match in every disease or condition; removing the selected genes from the analysis; and repeating the process of gene expression value selection for genes that cluster in a sub-fraction of the diseases or conditions; and iteratively repeating the generation of modules for each clusters until all gene clusters are exhausted. Examples of transcriptional modules for use with the present invention may be selected from:

Transcriptional Modules

Plasma cells: Includes genes encoding for Immunoglobulin chains (e.g. IGHM, IGJ, IGLL1, IGKC, IGHD) and the plasma cell marker CD38.;

Platelets: Includes genes encoding for platelet glycoproteins (ITGA2B, ITGB3, GP6, GPLL1A/B), and platelet-derived immune mediators such as PPPB (pro-platelet basic protein) and PF4 (platelet factor 4);

B-cells: Includes genes encoding for B-cell surface markers (CD72, CD79A/B, CD19, CD22) and other B-cell associated molecules: Early B-cell factor (EBF), B-cell linker (BLNK) and B lymphoid tyrosine kinase (BLK);

Undetermined. This set includes genes encoding regulators and targets of cAMP signaling pathway (JUND, ATF4, CREM, PDE4, NR4A2, VIL2), as well as repressors of TNF-alpha mediated NF-KB activation (CYLD, ASK, TNFAIP3);

Myeloid lineage: Includes genes encoding molecules expressed by cells of the myeloid lineage (CD86, CD163, FCGR2A), some of which being involved in pathogen recognition (CD14, TLR2, MYD88). This set also includes TNF family members (TNFR2, BAFF);

Undetermined. This set includes genes encoding for signaling molecules, e.g. the zinc finger containing inhibitor of activated STAT (PIAS1 and PIAS2), or the nuclear factor of activated T-cells NFATC3;

MHC/Ribosomal proteins: Almost exclusively formed by genes encoding MHC class I molecules (HLA-A,B,C,G,E)+ eta 2-microglobulin (B2M) or Ribosomal proteins (RPLs, RPSs);

Undetermined. Includes genes encoding metabolic enzymes (GLS, NSF1, NAT1) and factors involved in DNA replication (PURA, TERF2, EIF2S1);

Cytotoxic cells: Includes genes encoding cytotoxic T-cells amd NK-cells surface markers (CD8A, CD2, CD160, NKG7, KLRs), cytolytic molecules (granzyme, perforin, granulysin), chemokines (CCL5, XCL1) and CTL/NK-cell associated molecules (CTSW);

Neutrophils: This set includes genes encoding innate molecules that are found in neutrophil granules (Lactotransferrin: LTF, defensin: DEAF1, Bacterial Permeability Increasing protein: BPI, Cathelicidin antimicrobial protein: CAMP);

Erythrocytes: Includes genes encoding hemoglobin genes (HGBs) and other erythrocyte-associated genes (erythrocytic alkirin: ANK1, Glycophorin C: GYPC, hydroxymethylbilane synthase: HMBS, erythroid associated factor: ERAF);

Ribosomal proteins: Including genes encoding ribosomal proteins (RPLs, RPSs), Eukaryotic Translation Elongation factor family members (EEFs) and Nucleolar proteins (NPM1, NOAL2, NAP1L1);

Undetermined. This module includes genes encoding immune-related (CD40, CD80, CXCL12, IFNA5, IL4R) as well as cytoskeleton-related molecules (Myosin, Dedicator of Cytokenesis, Syndecan 2, Plexin C1, Distrobrevin);

Myeloid lineage: Related to M 1.5. Includes genes encoding genes expressed in myeloid lineage cells (IGTB2/CD18, Lymphotoxin beta receptor, Myeloid related proteins 8/14 Formyl peptide receptor 1), such as Monocytes and Neutrophils;

Undetermined. This module is largely composed of transcripts with no known function. Only 20 genes associated with literature, including a member of the chemokine-like factor superfamily (CKLFSF8);

T-cells: Includes genes encoding T-cell surface markers (CD5, CD6, CD7, CD26, CD28, CD96) and molecules expressed by lymphoid lineage cells (lymphotoxin beta, IL2-inducible T-cell kinase, TCF7, T-cell differentiation protein mal, GATA3, STAT5B);

Undetermined. Includes genes encoding molecules that associate to the cytoskeleton (Actin related protein ⅔, MAPK1, MAP3K1, RAB5A). Also present are T-cell expressed genes (FAS, ITGA4/CD49D, ZNF1A1);

Undetermined. Includes genes encoding for Immune-related cell surface molecules (CD36, CD86, LILRB), cytokines (IL15) and molecules involved in signaling pathways (FYB, TICAM2-Toll-like receptor pathway);

Undetermined. Includes genes encoding kinases (UHMK1, CSNK1G1, CDK6, WNK1, TAOK1, CALM2, PRKCI, ITPKB, SRPK2, STK17B, DYRK2, PIK3R1, STK4, CLK4, PKN2) and RAS family members (G3BP, RAB14, RASA2, RAP2A, KRAS);

Interferon-inducible: This set includes genes encoding interferon-inducible genes: antiviral molecules (OAS1/2/3/L, GBP1, G1P2, EIF2AK2/PKR, MX1, PML), chemokines (CXCL10/IP-10), signaling molecules (STAT1, STAt2, IRF7, ISGF3G);

Inflammation I: Includes genes encoding molecules involved in inflammatory processes (e.g. IL8, ICAM1, C5R1, CD44, PLAUR, IL1A, CXCL16), and regulators of apoptosis (MCL1, FOXO3A, RARA, BCL3/6/2A1, GADD45B);

Inflammation II: Includes genes encoding molecules inducing or inducible by Granulocyte-Macrophage CSF (SP11, IL18, ALOX5, ANPEP), as well as lysosomal enzymes (PPT1, CTSB/S, CES1, NEU1, ASAH1, LAMP2, CAST);

Undetermined. Includes genes encoding protein phosphates (PPP1R12A, PTPRC, PPP1CB, PPM1B) and phosphoinositide 3-kinase (PI3K) family members (PIK3CA, PIK32A, PIP5K3);

Undetermined. Composed of only a small number of transcripts. Includes genes encoding hemoglobin genes (HBA1, HBA2, HBB);

Undetermined. This very large set includes genes encoding T-cell surface markers (CD101, CD102, CD103) as well as molecules ubiquitously expressed among blood leukocytes (CXRCR1: fraktalkine receptor, CD47, P-selectin ligand);

Undetermined. Includes genes encoding proteasome subunits (PSMA2/5, PSMB5/8); ubiquitin protein ligases HIP2, STUB1, as well as components of ubiqutin ligase complexes (SUGT1);

Undetermined. Includes genes encoding for several enzymes: aminomethyltransferase, arginyltransferase, asparagines synthetase, diacylglycerol kinase, inositol phosphatases, methyltransferases, helicases; and

Undetermined. Includes genes encoding for protein kinases (PRKPIR, PRKDC, PRKCI) and phosphatases (e.g. PTPLB, PPP1R8/2CB). Also includes RAS oncogene family members and the NK cell receptor 2B4 (CD244);

and combinations thereof, wherein the level of expression of genes in a sample is charted to the modules to determine a disease or condition.

The present invention also includes a disease analysis tool that includes one or more gene modules selected from the group consisting of, for example,

Transcriptional Modules

Plasma cells: Includes genes encoding for Immunoglobulin chains (e.g. IGHM, IGJ, IGLL1, IGKC, IGHD) and the plasma cell marker CD38.;

Platelets: Includes genes encoding for platelet glycoproteins (ITGA2B, ITGB3, GP6, GP1A/B), and platelet-derived immune mediators such as PPPB (pro-platelet basic protein) and PF4 (platelet factor 4);

B-cells: Includes genes encoding for B-cell surface markers (CD72, CD79A/B, CD19, CD22) and other B-cell associated molecules: Early B-cell factor (EBF), B-cell linker (BLNK) and B lymphoid tyrosine kinase (BLK);

Undetermined. This set includes regulators and targets of cAMP signaling pathway (JUND, ATF4, CREM, PDE4, NR4A2, VIL2), as well as repressors of TNF-alpha mediated NF-KB activation (CYLD, ASK, TNFAIP3);

Myeloid lineage: Includes molecules expressed by cells of the myeloid lineage (CD86, CD163, FCGR2A), some of which being involved in pathogen recognition (CD14, TLR2, MYD88). This set also includes TNF family members (TNFR2, BAFF);

Undetermined. This set includes genes encoding for signaling molecules, e.g. the zinc finger containing inhibitor of activated STAT (PIAS1 and PIAS2), or the nuclear factor of activated T-cells NFATC3;

MHC/Ribosomal proteins: Almost exclusively formed by genes encoding MHC class I molecules (HLA-A,B,C,G,E)+ Beta 2-microglobulin (B2M) or Ribosomal proteins (RPLs, RPSs);

Undetermined. Includes genes encoding metabolic enzymes (GLS, NSF1, NAT1) and factors involved in DNA replication (PURA, TERF2, EIF2S1);

Cytotoxic cells: Includes cytotoxic T-cells amd NK-cells surface markers (CD8A, CD2, CD160, NKG7, KLRs), cytolytic molecules (granzyme, perforin, granulysin), chemokines (CCL5, XCL1) and CTL/NK-cell associated molecules (CTSW);

Neutrophils: This set includes innate molecules that are found in neutrophil granules (Lactotransferrin: LTF, defensin: DEAF1, Bacterial Permeability Increasing protein: BPI, Cathelicidin antimicrobial protein: CAMP . . . );

Erythrocytes: Includes hemoglobin genes (HGBs) and other erythrocyte-associated genes (erythrocytic alkirin: ANK1, Glycophorin C: GYPC, hydroxymethylbilane synthase: HMBS, erythroid associated factor: ERAF);

Ribosomal proteins: Including genes encoding ribosomal proteins (RPLs, RPSs), Eukaryotic Translation Elongation factor family members (EEFs) and Nucleolar proteins (NPM1, NOAL2, NAP1L1);

Undetermined. This module includes genes encoding immune-related (CD40, CD80, CXCL12, IFNA5, IL4R) as well as cytoskeleton-related molecules (Myosin, Dedicator of Cytokenesis, Syndecan 2, Plexin C1, Distrobrevin);

Myeloid lineage: Related to M 1.5. Includes genes expressed in myeloid lineage cells (IGTB2/CD18, Lymphotoxin beta receptor, Myeloid related proteins 8/14 Formyl peptide receptor 1), such as Monocytes and Neutrophils;

Undetermined. This module is largely composed of transcripts with no known function. Only 20 genes associated with literature, including a member of the chemokine-like factor superfamily (CKLFSF8);

T-cells: Includes T-cell surface markers (CD5, CD6, CD7, CD26, CD28, CD96) and molecules expressed by lymphoid lineage cells (lymphotoxin beta, IL2-inducible T-cell kinase, TCF7, T-cell differentiation protein mal, GATA3, STAT5B);

Undetermined. Includes genes encoding molecules that associate to the cytoskeleton (Actin related protein 2/3, MAPK1, MAP3K1, RAB5A). Also present are T-cell expressed genes (FAS, ITGA4/CD49D, ZNF1A1);

Undetermined. Includes genes encoding for Immune-related cell surface molecules (CD36, CD86, LILRB), cytokines (IL15) and molecules involved in signaling pathways (FYB, TICAM2-Toll-like receptor pathway);

Undetermined. Includes kinases (UHMK1, CSNK1G1, CDK6, WNK1, TAOK1, CALM2, PRKCI, ITPKB, SRPK2, STK17B, DYRK2, PIK3R1, STK4, CLK4, PKN2) and RAS family members (G3BP, RAB14, RASA2, RAP2A, KRAS);

Interferon-inducible: This set includes interferon-inducible genes: antiviral molecules (OAS1/2/3/L, GBP1, G1P2, EIF2AK2/PKR, MX1, PML), chemokines (CXCL10/IP-10), signaling molecules (STAT1, STAt2, IRF7, ISGF3G);

Inflammation I: Includes genes encoding molecules involved in inflammatory processes (e.g. IL8, ICAM1, C5R1, CD44, PLAUR, IL1A, CXCL16), and regulators of apoptosis (MCL1, FOXO3A, RARA, BCL3/6/2A1, GADD45B);

Inflammation II: Includes molecules inducing or inducible by Granulocyte-Macrophage CSF (SP11, IL18, ALOX5, ANPEP), as well as lysosomal enzymes (PPT1, CTSB/S, CES1, NEU1, ASAH1, LAMP2, CAST);

Undetermined. Includes protein phosphates (PPP1R12A, PTPRC, PPP1CB, PPM1B) and phosphoinositide 3-kinase (PI3K) family members (PIK3CA, PIK32A, PIP5K3);

Undetermined. Composed of only a small number of transcripts. Includes hemoglobin genes (HBA1, HBA2, HBB);

Undetermined. This very large set includes T-cell surface markers (CD101, CD102, CD103) as well as molecules ubiquitously expressed among blood leukocytes (CXRCR1: fraktalkine receptor, CD47, P-selectin ligand);

Undetermined. Includes genes encoding proteasome subunits (PSMA2/5, PSMB5/8); ubiquitin protein ligases HIP2, STUB1, as well as components of ubiqutin ligase complexes (SUGT1);

Undetermined. Includes genes encoding for several enzymes: aminomethyltransferase, arginyltransferase, asparagines synthetase, diacylglycerol kinase, inositol phosphatases, methyltransferases, helicases; and

Undetermined. Includes genes encoding for protein kinases (PRKPIR, PRKDC, PRKCI) and phosphatases (e.g. PTPLB, PPP1R8/2CB). Also includes RAS oncogene family members and the NK cell receptor 2B4 (CD244);

sufficient to distinguish between an autoimmune disease, a viral infection a bacterial infection, cancer and transplant rejection. The modules are used to distinguish between Systemic Lupus erythematosus, Influenza infection, melanoma and transplant rejection.

In one embodiment, the modules selected may be selected from:

Plasma cells: Includes genes encoding for Immunoglobulin chains (e.g. IGHM, IGJ, IGLL1, IGKC, IGHD) and the plasma cell marker CD38; and

Platelets: Includes genes encoding for platelet glycoproteins (ITGA2B, ITGB3, GP6, GP1A/B), and platelet-derived immune mediators such as PPPB (pro-platelet basic protein) and PF4 (platelet factor 4);

and the modules are used to identify Systemic Lupus erythematosus by having a positive vector at these two modules.

In another embodiment, the modules selected may be selected from:

Plasma cells: Includes genes encoding for Immunoglobulin chains (e.g. IGHM, IGJ, IGLL1, IGKC, IGHD) and the plasma cell marker CD38; and

Platelets: Includes genes encoding for platelet glycoproteins (ITGA2B, ITGB3, GP6, GP1A/B), and platelet-derived immune mediators such as PPPB (pro-platelet basic protein) and PF4 (platelet factor 4);

and the modules are used to identify Influenza infection by having neither a positive nor a negative vector at these two modules.

In another embodiment, the modules selected may be selected from:

Plasma cells: Includes genes encoding for Immunoglobulin chains (e.g. IGHM, IGJ, IGLL1, IGKC, IGHD) and the plasma cell marker CD38; and

Platelets: Includes genes encoding for platelet glycoproteins (ITGA2B, ITGB3, GP6, GP1A/B), and platelet-derived immune mediators such as PPPB (pro-platelet basic protein) and PF4 (platelet factor 4);

and the modules are used to identify melanoma by having a negative vector for the plasma cell markers and a positive vector for the platelet markers.

In another embodiment, the modules selected may be selected from:

Plasma cells: Includes genes encoding for Immunoglobulin chains (e.g. IGHM, IGJ, IGLL1, IGKC, IGHD) and the plasma cell marker CD38; and

Platelets: Includes genes encoding for platelet glycoproteins (ITGA2B, ITGB3, GP6, GP1A/B), and platelet-derived immune mediators such as PPPB (pro-platelet basic protein) and PF4 (platelet factor 4);

and the modules are used to identify transplant rejection by having a negative vectors at these two modules.

In another embodiment, the modules selected may be selected from:

Plasma cells: Includes genes encoding for Immunoglobulin chains (e.g. IGHM, IGJ, IGLL1, IGKC, IGHD) and the plasma cell marker CD38; and

Platelets: Includes genes encoding for platelet glycoproteins (ITGA2B, ITGB3, GP6, GP1A/B), and platelet-derived immune mediators such as PPPB (pro-platelet basic protein) and PF4 (platelet factor 4);

and the modules are used to identify Influenza infection by having a negative vector at these two modules.

Yet another embodiment of the present invention is a prognostic gene array that includes a customized gene array that has a combination of genes that are representative of one or more transcriptional modules, wherein the transcriptome of a patient that is contacted with the customized gene array is prognostic of one or more disease or conditions that match the transcriptional modules. In one example, the patient's immune response to the disease or condition is determined based on the presence, absence or level of expression of genes of the transcriptome based on a correlation of the transcriptional modules with a specific disease or condition. The array can distinguish between an autoimmune disease, a viral infection a bacterial infection, cancer and transplant rejection. The array may even be organized into two or more transcriptional modules. For example, the array may be organized into three transcriptional modules that include one or more submodules selected from:

| | Number | | |
|---|---|---|---|
| | of probe | | |
| Submodule | sets | Keyword selection | Assessment |
| M 1.1 | 69 | Ig, | Plasma cells: Includes genes encoding for |
| | | Immunoglobulin, | Immunoglobulin chains (e.g. IGHM, IGJ, IGLL1, |
| | | Bone, Marrow, | IGKC, IGHD) and the plasma cell marker CD38; |
| | | PreB, IgM, Mu. | |
| M 1.2 | 96 | Platelet, Adhesion, | Platelets: Includes genes encoding for platelet |
| | | Aggregation, | glycoproteins (ITGA2B, ITGB3, GP6, GP1A/B), and |
| | | Endothelial, | platelet-derived immune mediators such as PPPB (pro- |
| | | Vascular | platelet basic protein) and PF4 (platelet factor 4); |
| M 1.3 | 47 | Immunoreceptor, | B-cells: Includes genes encoding for B-cell surface |
| | | BCR, B-cell, IgG | markers (CD72, CD79A/B, CD19, CD22) and other |
| | | | B-cell associated molecules: Early B-cell factor (EBF), |
| | | | B-cell linker (BLNK) and B lymphoid tyrosine kinase |
| | | | (BLK); |
| M 1.4 | 87 | Replication, | Undetermined. This set includes regulators and targets |
| | | Repression Repair, | of cAMP signaling pathway (JUND, ATF4, CREM, |
| | | CREB, Lymphoid, | PDE4, NR4A2, VIL2), as well as repressors of TNF- |
| | | TNF-alpha | alpha mediated NF-KB activation (CYLD, ASK, |
| | | | TNFAIP3); |
| M 1.5 | 130 | Monocytes, | Myeloid lineage: Includes molecules expressed by |
| | | Dendritic, MHC, | cells of the myeloid lineage (CD86, CD163, |
| | | Costimulatory, | FCGR2A), some of which being involved in pathogen |
| | | TLR4, MYD88 | recognition (CD14, TLR2, MYD88). This set also |
| | | | includes TNF family members (TNFR2, BAFF); |
| M 1.6 | 28 | Zinc, Finger, P53, | Undetermined. This set includes genes encoding for |
| | | RAS | signaling molecules, e.g. the zinc finger containing |
| | | | inhibitor of activated STAT (PIAS1 and PIAS2), or |
| | | | the nuclear factor of activated T-cells NFATC3; |
| M 1.7 | 127 | Ribosome, | MHC/Ribosomal proteins: Almost exclusively formed |
| | | Translational, 40S, | by genes encoding MHC class I molecules (HLA- |
| | | 60S, HLA | A,B,C,G,E) + Beta 2-microglobulin (B2M) or |
| | | | Ribosomal proteins (RPLs, RPSs); |
| M 1.8 | 86 | Metabolism, | Undetermined. Includes genes encoding metabolic |
| | | Biosynthesis, | enzymes (GLS, NSF1, NAT1) and factors involved in |
| | | Replication, | DNA replication (PURA, TERF2, EIF2S1); |
| | | Helicase | |
| M 2.1 | 72 | NK, Killer, | Cytotoxic cells: Includes cytotoxic T-cells and NK- |
| | | Cytolytic, CD8, | cells surface markers (CD8A, CD2, CD160, NKG7, |
| | | Cell-mediated, T- | KLRs), cytolytic molecules (granzyme, perforin, |
| | | cell, CTL, IFN-g | granulysin), chemokines (CCL5, XCL1) and CTL/NK- |
| | | | cell associated molecules (CTSW); |
| M 2.2 | 44 | Granulocytes, | Neutrophils: This set includes innate molecules that |
| | | Neutrophils, | are found in neutrophil granules (Lactotransferrin: |
| | | Defense, Myeloid, | LTF, defensin: DEAF1, Bacterial Permeability |
| | | Marrow | Increasing protein: BPI, Cathelicidin antimicrobial |
| | | | protein: CAMP); |
| M 2.3 | 94 | Erythrocytes, Red, | Erythrocytes: Includes hemoglobin genes (HGBs) and |
| | | Anemia, Globin, | other erythrocyte-associated genes (erythrocytic |
| | | Hemoglobin | alkirin:ANK1, Glycophorin C: GYPC, |
| | | | hydroxymethylbilane synthase: HMBS, erythroid |
| | | | associated factor: ERAF); |
| M 2.4 | 118 | Ribonucleoprotein, | Ribosomal proteins: Including genes encoding |
| | | 60S, nucleolus, | ribosomal proteins (RPLs, RPSs), Eukaryotic |
| | | Assembly, | Translation Elongation factor family members (EEFs) |
| | | Elongation | and Nucleolar proteins (NPM1, NOAL2, NAP1L1); |
| M 2.5 | 242 | Adenoma, | Undetermined. This module includes genes encoding |
| | | Interstitial, | immune-related (CD40, CD80, CXCL12, IFNA5, |
| | | Mesenchyme, | IL4R) as well as cytoskeleton-related molecules |
| | | Dendrite, Motor | (Myosin, Dedicator of Cytokenesis, Syndecan 2, |
| | | | Plexin C1, Distrobrevin); |
| M 2.6 | 110 | Granulocytes, | Myeloid lineage: Related to M 1.5. Includes genes |
| | | Monocytes, | expressed in myeloid lineage cells (IGTB2/CD18, |
| | | Myeloid, ERK, | Lymphotoxin beta receptor, Myeloid related proteins |
| | | Necrosis | 8/14 Formyl peptide receptor 1), such as Monocytes |
| | | | and Neutrophils; |
| M 2.7 | 43 | No keywords | Undetermined. This module is largely composed of |
| | | extracted. | transcripts with no known function. Only 20 genes |
| | | | associated with literature, including a member of the |
| | | | chemokine-like factor superfamily (CKLFSF8); |
| M 2.8 | 104 | Lymphoma, T-cell, | T-cells: Includes T-cell surface markers (CD5, CD6, |
| | | CD4, CD8, TCR, | CD7, CD26, CD28, CD96) and molecules expressed |
| | | Thymus, | by lymphoid lineage cells (lymphotoxin beta, IL2- |
| | | Lymphoid, IL2 | inducible T-cell kinase, TCF7, T-cell differentiation |
| | | | protein mal, GATA3, STAT5B); |
| M 2.9 | 122 | ERK, | Undetermined. Includes genes encoding molecules |
| | | Transactivation, | that associate to the cytoskeleton (Actin related protein |
| | | Cytoskeletal, | 2/3, MAPK1, MAP3K1, RAB5A). Also present are T- |
| | | MAPK, JNK | cell expressed genes (FAS, ITGA4/CD49D, ZNF1A1); |
| M 2.10 | 44 | Myeloid, | Undetermined. Includes genes encoding for Immune- |
| | | Macrophage, | related cell surface molecules (CD36, CD86, LILRB), |
| | | Dendritic, | cytokines (IL15) and molecules involved in signaling |
| | | Inflammatory, | pathways (FYB, TICAM2-Toll-like receptor |
| | | Interleukin | pathway); |
| M 2.11 | 77 | Replication, | Undetermined. Includes kinases (UHMK1, CSNK1G1, |
| | | Repress, RAS, | CDK6, WNK1, TAOK1, CALM2, PRKCI, ITPKB, |
| | | Autophosphorylation, | SRPK2, STK17B, DYRK2, PIK3R1, STK4, CLK4, |
| | | Oncogenic | PKN2) and RAS family members (G3BP, RAB14, |
| | | | RASA2, RAP2A, KRAS); |
| M 3.1 | 80 | ISRE, Influenza, | Interferon-inducible: This set includes interferon- |
| | | Antiviral, IFN- | inducible genes: antiviral molecules (OAS1/2/3/L, |
| | | gamma, IFN-alpha, | GBP1, G1P2, EIF2AK2/PKR, MX1, PML), |
| | | Interferon | chemokines (CXCL10/IP-10), signaling molecules |
| | | | (STAT1, STAt2, IRF7, ISGF3G); |
| M 3.2 | 230 | TGF-beta, TNF, | Inflammation I: Includes genes encoding molecules |
| | | Inflammatory, | involved in inflammatory processes (e.g. IL8, ICAM1, |
| | | Apoptotic, | C5R1, CD44, PLAUR, IL1A, CXCL16), and |
| | | Lipopolysaccharide | regulators of apoptosis (MCL1, FOXO3A, RARA, |
| | | | BCL3/6/2A1, GADD45B); |
| M 3.3 | 230 | Granulocyte, | Inflammation II: Includes molecules inducing or |
| | | Inflammatory, | inducible by Granulocyte-Macrophage CSF (SPI1, |
| | | Defense, Oxidize, | IL18, ALOX5, ANPEP), as well as lysosomal |
| | | Lysosomal | enzymes (PPT1, CTSB/S, CES1, NEU1, ASAH1, |
| | | | LAMP2, CAST); |
| M 3.4 | 323 | No keyword | Undetermined. Includes protein phosphates |
| | | extracted | (PPP1R12A, PTPRC, PPP1CB, PPM1B) and |
| | | | phosphoinositide 3-kinase (PI3K) family members |
| | | | (PIK3CA, PIK32A, PIP5K3); |
| M 3.5 | 19 | No keyword | Undetermined. Composed of only a small number of |
| | | extracted | transcripts. Includes hemoglobin genes (HBA1, |
| | | | HBA2, HBB); |
| M 3.6 | 233 | Complement, Host, | Undetermined. This very large set includes T-cell |
| | | Oxidative, | surface markers (CD101, CD102, CD103) as well as |
| | | Cytoskeletal, T- | molecules ubiquitously expressed among blood |
| | | cell | leukocytes (CXRCR1: fraktalkine receptor, CD47, P- |
| | | | selectin ligand); |
| M 3.7 | 80 | Spliceosome, | Undetermined. Includes genes encoding proteasome |
| | | Methylation, | subunits (PSMA2/5, PSMB5/8); ubiquitin protein |
| | | Ubiquitin, Beta- | ligases HIP2, STUB1, as well as components of |
| | | catenin | ubiqutin ligase complexes (SUGT1); |
| M 3.8 | 182 | CDC, TCR, CREB, | Undetermined. Includes genes encoding for several |
| | | Glycosylase | enzymes: aminomethyltransferase, arginyltransferase, |
| | | | asparagines synthetase, diacylglycerol kinase, inositol |
| | | | phosphatases, methyltransferases, helicases; and |
| M 3.9 | 261 | Chromatin, | Undetermined. Includes genes encoding for protein |
| | | Checkpoint, | kinases (PRKPIR, PRKDC, PRKCI) and phosphatases |
| | | Replication, | (e.g. PTPLB, PPP1R8/2CB). Also includes RAS |
| | | Transactivation | oncogene family members and the NK cell receptor |
| | | | 2B4 (CD244); |

wherein one or more probes from each that bind specifically one or more of the genes in the module.

Yet another invention includes a gene analysis tool that includes one or more gene modules selected from a combination of one group selected from the left column and one group selected from the right column including:

| Keyword selection | Transcriptional modules |
|---|---|
| Ig, Immunoglobulin, | Plasma cells: Includes genes encoding for Immunoglobulin chains (e.g. IGHM, |
| Bone, Marrow, PreB, | IGJ, IGLL1, IGKC, IGHD) and the plasma cell marker CD38.; |
| IgM, Mu. | |
| Platelet, Adhesion, | Platelets: Includes genes encoding for platelet glycoproteins (ITGA2B, ITGB3, |
| Aggregation, | GP6, GP1A/B), and platelet-derived immune mediators such as PPPB (pro- |
| Endothelial, Vascular | platelet basic protein) and PF4 (platelet factor 4); |
| Immunoreceptor, | B-cells: Includes genes encoding for B-cell surface markers (CD72, CD79A/B, |
| BCR, B-cell, IgG | CD19, CD22) and other B-cell associated molecules: Early B-cell factor (EBF), |
| | B-cell linker (BLNK) and B lymphoid tyrosine kinase (BLK); |
| Replication, | Undetermined. This set includes regulators and targets of cAMP signaling |
| Repression, Repair, | pathway (JUND, ATF4, CREM, PDE4, NR4A2, VIL2), as well as repressors of |
| CREB, Lymphoid, | TNF-alpha mediated NF-KB activation (CYLD, ASK, TNFAIP3); |
| TNF-alpha | |
| Monocytes, | Myeloid lineage: Includes molecules expressed by cells of the myeloid lineage |
| Dendritic, MHC, | (CD86, CD163, FCGR2A), some of which being involved in pathogen |
| Costimulatory, | recognition (CD14, TLR2, MYD88). This set also includes TNF family |
| TLR4, MYD88 | members (TNFR2, BAFF); |
| Zinc, Finger, P53, | Undetermined. This set includes genes encoding for signaling molecules, e.g. |
| RAS | the zinc finger containing inhibitor of activated STAT (PIAS1 and PIAS2), or |
| | the nuclear factor of activated T-cells NFATC3; |
| Ribosome, | MHC/Ribosomal proteins: Almost exclusively formed by genes encoding MHC |
| Translational, 40S, | class I molecules (HLA-A,B,C,G,E) + Beta 2-microglobulin (B2M) or |
| 60S, HLA | Ribosomal proteins (RPLs, RPSs); |
| Metabolism, | Undetermined. Includes genes encoding metabolic enzymes (GLS, NSF1, |
| Biosynthesis, | NAT1) and factors involved in DNA replication (PURA, TERF2, EIF2S1); |
| Replication, Helicase | |
| NK, Killer, | Cytotoxic cells: Includes cytotoxic T-cells amd NK-cells surface markers |
| Cytolytic, CD8, Cell- | (CD8A, CD2, CD160, NKG7, KLRs), cytolytic molecules (granzyme, perforin, |
| mediated, T-cell, | granulysin), chemokines (CCL5, XCL1) and CTL/NK-cell associated molecules |
| CTL, IFN-g | (CTSW); |
| Granulocytes, | Neutrophils: This set includes innate molecules that are found in neutrophil |
| Neutrophils, | granules (Lactotransferrin: LTF, defensin: DEAF1, Bacterial Permeability |
| Defense, Myeloid, | Increasing protein: BPI, Cathelicidin antimicrobial protein: CAMP . . . ); |
| Marrow | |
| Erythrocytes, Red, | Erythrocytes: Includes hemoglobin genes (HGBs) and other erythrocyte- |
| Anemia, Globin, | associated genes (erythrocytic alkirin:ANK1, Glycophorin C: GYPC, |
| Hemoglobin | hydroxymethylbilane synthase: HMBS, erythroid associated factor: ERAF); |
| Ribonucleoprotein, | Ribosomal proteins: Including genes encoding ribosomal proteins (RPLs, RPSs), |
| 60S, nucleolus, | Eukaryotic Translation Elongation factor family members (EEFs) and Nucleolar |
| Assembly, | proteins (NPM1, NOAL2, NAP1L1); |
| Elongation | |
| Adenoma, | Undetermined. This module includes genes encoding immune-related (CD40, |
| Interstitial, | CD80, CXCL12, IFNA5, IL4R) as well as cytoskeleton-related molecules |
| Mesenchyme, | (Myosin, Dedicator of Cytokenesis, Syndecan 2, Plexin C1, Distrobrevin); |
| Dendrite, Motor | |
| Granulocytes, | Myeloid lineage: Related to M 1.5. Includes genes expressed in myeloid lineage |
| Monocytes, Myeloid, | cells (IGTB2/CD18, Lymphotoxin beta receptor, Myeloid related proteins 8/14 |
| ERK, Necrosis | Formyl peptide receptor 1), such as Monocytes and Neutrophils; |
| No keywords | Undetermined. This module is largely composed of transcripts with no known |
| extracted. | function. Only 20 genes associated with literature, including a member of the |
| | chemokine-like factor superfamily (CKLFSF8); |
| Lymphoma, T-cell, | T-cells: Includes T-cell surface markers (CD5, CD6, CD7, CD26, CD28, CD96) |
| CD4, CD8, TCR, | and molecules expressed by lymphoid lineage cells (lymphotoxin beta, IL2- |
| Thymus, Lymphoid, | inducible T-cell kinase, TCF7, T-cell differentiation protein mal, GATA3, |
| IL2 | STAT5B); |
| ERK, | Undetermined. Includes genes encoding molecules that associate to the |
| Transactivation, | cytoskeleton (Actin related protein 2/3, MAPK1, MAP3K1, RAB5A). Also |
| Cytoskeletal, MAPK, | present are T-cell expressed genes (FAS, ITGA4/CD49D, ZNF1A1); |
| JNK | |
| Myeloid, | Undetermined. Includes genes encoding for Immune-related cell surface |
| Macrophage, | molecules (CD36, CD86, LILRB), cytokines (IL15) and molecules involved in |
| Dendritic, | signaling pathways (FYB, TICAM2-Toll-like receptor pathway); |
| Inflammatory, | |
| Interleukin | |
| Replication, Repress, | Undetermined. Includes kinases (UHMK1, CSNK1G1, CDK6, WNK1, TAOK1, |
| RAS, | CALM2, PRKCI, ITPKB, SRPK2, STK17B, DYRK2, PIK3R1, STK4, CLK4, |
| Autophosphorylation, | PKN2) and RAS family members (G3BP, RAB14, RASA2, RAP2A, KRAS); |
| Oncogenic | |
| ISRE, Influenza, | Interferon-inducible: This set includes interferon-inducible genes: antiviral |
| Antiviral, IFN- | molecules (OAS1/2/3/L, GBP1, G1P2, EIF2AK2/PKR, MX1, PML), |
| gamma, IFN-alpha, | chemokines (CXCL10/IP-10), signaling molecules (STAT1, STAt2, IRF7, |
| Interferon | ISGF3G); |
| TGF-beta, TNF, | Inflammation I: Includes genes encoding molecules involved in inflammatory |
| Inflammatory, | processes (e.g. IL8, ICAM1, C5R1, CD44, PLAUR, IL1A, CXCL16), and |
| Apoptotic, | regulators of apoptosis (MCL1, FOXO3A, RARA, BCL3/6/2A1, GADD45B); |
| Lipopolysaccharide | |
| Granulocyte, | Inflammation II: Includes molecules inducing or inducible by Granulocyte- |
| Inflammatory, | Macrophage CSF (SPI1, IL18, ALOX5, ANPEP), as well as lysosomal enzymes |
| Defense, Oxidize, | (PPT1, CTSB/S, CES1, NEU1, ASAH1, LAMP2, CAST); |
| Lysosomal | |
| No keyword | Undetermined. Includes protein phosphates (PPP1R12A, PTPRC, PPP1CB, |
| extracted | PPM1B) and phosphoinositide 3-kinase (PI3K) family members (PIK3CA, |
| | PIK32A, PIP5K3); |
| No keyword | Undetermined. Composed of only a small number of transcripts. Includes |
| extracted | hemoglobin genes (HBA1, HBA2, HBB); |
| Complement, Host, | Undetermined. This very large set includes T-cell surface markers (CD101, |
| Oxidative, | CD102, CD103) as well as molecules ubiquitously expressed among blood |
| Cytoskeletal, T-cell | leukocytes (CXRCR1: fraktalkine receptor, CD47, P-selectin ligand); |
| Spliceosome, | Undetermined. Includes genes encoding proteasome subunits (PSMA2/5, |
| Methylation, | PSMB5/8); ubiquitin protein ligases HIP2, STUB1, as well as components of |
| Ubiquitin, Beta- | ubiqutin ligase complexes (SUGT1); |
| catenin | |
| CDC, TCR, CREB, | Undetermined. Includes genes encoding for several enzymes: |
| Glycosylase | aminomethyltransferase, arginyltransferase, asparagines synthetase, |
| | diacylglycerol kinase, inositol phosphatases, methyltransferases, helicases; and |
| Chromatin, | Undetermined. Includes genes encoding for protein kinases (PRKPIR, PRKDC, |
| Checkpoint, | PRKCI) and phosphatases (e.g. PTPLB, PPP1R8/2CB). Also includes RAS |
| Replication, | oncogene family members and the NK cell receptor 2B4 (CD244); |
| Transactivation | |

and combinations thereof, wherein the level of expression of genes in a sample is charted to the modules to determine a disease or condition.

The arrays, methods and systems of the present invetnion may even be used to select patients for a clinical trial by obtaining the transcriptome of a prospective patient; comparing the transcriptome to one or more transcriptional modules that are indicative of a disease or condition that is to be treated in the clinical trial; and determining the likelihood that a patient is a good candidate for the clinical trial based on the presence, absence or level of one or more genes that are expressed in the patient's transcriptome within one or more transcriptional modules that are correlated with success in a clinical trial. Generally, for each module a vector that correlates with a sum of the proportion of transcripts in a sample may be used, e.g., when each module includes a vector and wherein one or more diseases or conditions is associated with the one or more vectors. Therefore, each module may include a vector that correlates to the expression level of one or more genes within each module.

The present invention also includes arrays, e.g., custom microarrays, that include nucleic acid probes immobilized on a solid support that includes sufficient probes from one or more modules to provide a sufficient proportion of differentially expressed genes to distinguish between one or more diseases, the probes being selected from Table 3. For example, an array of nucleic acid probes immobilized on a solid support, in which the array includes at least two sets of probe modules selected from:

| Module | |
|---|---|
| I.D. | Transcriptional Modules |
| M 1.1 | Plasma cells: Includes genes encoding for Immunoglobulin chains (e.g. IGHM, IGJ, |
| | IGLL1, IGKC, IGHD) and the plasma cell marker CD38. |
| M 1.2 | Platelets: Includes genes encoding for platelet glycoproteins (ITGA2B, ITGB3, GP6, |
| | GP1A/B), and platelet-derived immune mediators such as PPPB (pro-platelet basic protein) |
| | and PF4 (platelet factor 4). |
| M 1.3 | B-cells: Includes genes encoding for B-cell surface markers (CD72, CD79A/B, CD19, |
| | CD22) and other B-cell associated molecules: Early B-cell factor (EBF), B-cell linker |
| | (BLNK) and B lymphoid tyrosine kinase (BLK). |
| M 1.4 | Undetermined. This set includes regulators and targets of cAMP signaling pathway |
| | (JUND, ATF4, CREM, PDE4, NR4A2, VIL2), as well as repressors of TNF-alpha |
| | mediated NF-KB activation (CYLD, ASK, TNFAIP3). |
| M 1.5 | Myeloid lineage: Includes molecules expressed by cells of the myeloid lineage (CD86, |
| | CD163, FCGR2A), some of which being involved in pathogen recognition (CD14, TLR2, |
| | MYD88). This set also includes TNF family members (TNFR2, BAFF). |
| M 1.6 | Undetermined. This set includes genes encoding for signaling molecules, e.g. the zinc |
| | finger containing inhibitor of activated STAT (PIAS1 and PIAS2), or the nuclear factor of |
| | activated T-cells NFATC3. |
| M 1.7 | MHC/Ribosomal proteins: Almost exclusively formed by genes encoding MHC class I |
| | molecules (HLA-A,B,C,G,E) + Beta 2-microglobulin (B2M) or Ribosomal proteins (RPLs, |
| | RPSs). |
| M 1.8 | Undetermined. Includes genes encoding metabolic enzymes (GLS, NSF1, NAT1) and |
| | factors involved in DNA replication (PURA, TERF2, EIF2S1). |
| M 2.1 | Cytotoxic cells: Includes cytotoxic T-cells amd NK-cells surface markers (CD8A, CD2, |
| | CD160, NKG7, KLRs), cytolytic molecules (granzyme, perforin, granulysin), chemokines |
| | (CCL5, XCL1) and CTL/NK-cell associated molecules (CTSW). |
| M 2.2 | Neutrophils: This set includes innate molecules that are found in neutrophil granules |
| | (Lactotransferrin: LTF, defensin: DEAF1, Bacterial Permeability Increasing protein: BPI, |
| | Cathelicidin antimicrobial protein: CAMP . . . ). |
| M 2.3 | Erythrocytes: Includes hemoglobin genes (HGBs) and other erythrocyte-associated genes |
| | (erythrocytic alkirin: ANK1, Glycophorin C: GYPC, hydroxymethylbilane synthase: |
| | HMBS, erythroid associated factor: ERAF). |
| M 2.4 | Ribosomal proteins: Including genes encoding ribosomal proteins (RPLs, RPSs), |
| | Eukaryotic Translation Elongation factor family members (EEFs) and Nucleolar proteins |
| | (NPM1, NOAL2, NAP1L1). |
| M 2.5 | Undetermined. This module includes genes encoding immune-related (CD40, CD80, |
| | CXCL12, IFNA5, IL4R) as well as cytoskeleton-related molecules (Myosin, Dedicator of |
| | Cytokenesis, Syndecan 2, Plexin C1, Distrobrevin). |
| M 2.6 | Myeloid lineage: Related to M 1.5. Includes genes expressed in myeloid lineage cells |
| | (IGTB2/CD18, Lymphotoxin beta receptor, Myeloid related proteins 8/14 Formyl peptide |
| | receptor 1), such as Monocytes and Neutrophils: |
| M 2.7 | Undetermined. This module is largely composed of transcripts with no known function. |
| | Only 20 genes associated with literature, including a member of the chemokine-like factor |
| | superfamily (CKLFSF8). |
| M 2.8 | T-cells: Includes T-cell surface markers (CD5, CD6, CD7, CD26, CD28, CD96) and |
| | molecules expressed by lymphoid lineage cells (lymphotoxin beta, IL2-inducible T-cell |
| | kinase, TCF7, T-cell differentiation protein mal, GATA3, STAT5B). |
| M 2.9 | Undetermined. Includes genes encoding molecules that associate to the cytoskeleton (Actin |
| | related protein 2/3, MAPK1, MAP3K1, RAB5A). Also present are T-cell expressed genes |
| | (FAS, ITGA4/CD49D, ZNF1A1). |
| M 2.10 | Undetermined. Includes genes encoding for Immune-related cell surface molecules (CD36, |
| | CD86, LILRB), cytokines (IL15) and molecules involved in signaling pathways (FYB, |
| | TICAM2-Toll-like receptor pathway). |
| M 2.11 | Undetermined. Includes kinases (UHMK1, CSNK1G1, CDK6, WNK1, TAOK1, CALM2, |
| | PRKCI, ITPKB, SRPK2, STK17B, DYRK2, PIK3R1, STK4, CLK4, PKN2) and RAS |
| | family members (G3BP, RAB14, RASA2, RAP2A, KRAS). |
| M 3.1 | Interferon-inducible: This set includes interferon-inducible genes: antiviral molecules |
| | (OAS1/2/3/L, GBP1, G1P2, EIF2AK2/PKR, MX1, PML), chemokines (CXCL10/IP-10), |
| | signaling molecules (STAT1, STAt2, IRF7, ISGF3G). |
| M 3.2 | Inflammation I: Includes genes encoding molecules involved in inflammatory processes |
| | (e.g. IL8, ICAM1, C5R1, CD44, PLAUR, IL1A, CXCL16), and regulators of apoptosis |
| | (MCL1, FOXO3A, RARA, BCL3/6/2A1, GADD45B). |
| M 3.3 | Inflammation II: Includes molecules inducing or inducible by Granulocyte-Macrophage |
| | CSF (SPI1, IL18, ALOX5, ANPEP), as well as lysosomal enzymes (PPT1, CTSB/S, |
| | CES1, NEU1, ASAH1, LAMP2, CAST). |
| M 3.4 | Undetermined. Includes protein phosphates (PPP1R12A, PTPRC, PPP1CB, PPM1B) and |
| | phosphoinositide 3-kinase (PI3K) family members (PIK3CA, PIK32A, PIP5K3). |
| M 3.5 | Undetermined. Composed of only a small number of transcripts. Includes hemoglobin |
| | genes (HBA1, HBA2, HBB). |
| M 3.6 | Undetermined. This very large set includes T-cell surface markers (CD101, CD102, |
| | CD103) as well as molecules ubiquitously expressed among blood leukocytes (CXRCR1: |
| | fraktalkine receptor, CD47, P-selectin ligand). |
| M 3.7 | Undetermined. Includes genes encoding proteasome subunits (PSMA2/5, PSMB5/8); |
| | ubiquitin protein ligases HIP2, STUB1, as well as components of ubiqutin ligase |
| | complexes (SUGT1). |
| M 3.8 | Undetermined. Includes genes encoding for several enzymes: aminomethyltransferase, |
| | arginyltransferase, asparagines synthetase, diacylglycerol kinase, inositol phosphatases, |
| | methyltransferases, helicases . . . |
| M 3.9 | Undetermined. Includes genes encoding for protein kinases (PRKPIR, PRKDC, PRKCI) |
| | and phosphatases (e.g. PTPLB, PPP1R8/2CB). Also includes RAS oncogene family |
| | members and the NK cell receptor 2B4 (CD244). |

wherein the probes in the first probe set have one or more interrogation positions respectively corresponding to one or more diseases. The array may have between 100 and 100,000 probes, and each probe may be, e.g., 9-21 nucleotides long. When separated into organized prose sets, these may be interrogated separately.

The present invention also includes one or more nucleic acid probes immobilized on a solid support to form a module array that includes at least one pair of first and second probe groups, each group having one or more probes as defined by Table 3. The probe groups are selected to provide a composite transcriptional marker vector that is consistent across microarray platforms. In fact, the probe groups may even be used to provide a composite transcriptional marker vector that is consistent across microarray platforms and displayed in a summary for regulatory approval. The skilled artisan will appreciate that using the modules of the present invention it is possible to rapidly develop one or more disease specific arrays that may be used to rapidly diagnose or distinguish between different disease and/or conditions.

### BRIEF DESCRIPTION OF THE DRAWINGS

For a more complete understanding of the features and advantages of the present invention, reference is now made to the detailed description of the invention along with the accompanying figures and in which:

FIG. 1A to 1C show the basic microarray data mining strategy steps involved in accepted gene-level microarray data analysis (FIG. 1A), the modular mining strategy of the present invention FIG. 1B and a full size representation of the module extraction algorithm FIG. 1C. FIG. 1C provides a more detailed view of the module extraction algorithm in which, in FIG. 1C-1, step (a) shows examples of data are generated in the context of a defined experimental system (e.g. ex-vivo PBMCs); step (b) shows that the transcriptional profiles are obtained for several experimental groups (e.g. S1-8); step (c) shows that for each group, genes are distributed among x clusters (e.g. x=30) based on similarity of expression profiles (using K-means clustering algorithms); and in FIG. 1C-2, step (d) shows the cluster distribution of each gene across the different experimental groups is recorded into a table and distribution patterns are matched; and step (e) shows that modules are selected through an iterative process, starting with the largest set of genes distributed among the same cluster across all experimental groups (are found in the same cluster for eight out of eight groups). The selection is expanded from this core reference pattern to include genes with ⅞, 6/8 and ⅝ matches. Once a module has been formed, the genes are withdrawn from the selection pool. The process is then repeated, starting with the second largest group of genes, progressively reducing levels of stringency.

FIG. 2 Modular gene expression profiles across an independent group of samples. Differences in transcriptional behavior between modules are illustrated in a set of samples obtained from twenty-one healthy volunteers. The samples were not used in the module selection process. The graphs represent transcriptional profiles, with each line showing levels of expression (y-axis) of a single transcript across multiple conditions (samples, x-axis). Transcriptional profiles of Modules 1.2, 1.7, 2.1 and 2.11 are shown. The expression of each gene is normalized to the median of the measurements obtained across all samples.

FIG. 3 Distribution of keyword occurrence in the literature obtained for four sets of coordinately expressed genes. Term occurrence levels in abstracts were computed for all the genes in M3.1, M1.5, M1.3 and M1.2 associated with at least ten publications (representing more than 26,000 abstracts). Keyword profiles were extracted for each module and a selection was used to generate this figure. Levels of keyword occurrence in abstracts are indicated by color scale, with yellow representing high occurrence. M3.1 is associated to interferon, M1.5 is associated to pathogen recognition molecules/myeloid lineage cells, M1.3 is associated with B-cells and M1.2 is associated with Platelets:

FIGS. 4A-B Modular microarray analysis strategy. The proposed microarray data analysis strategy includes two basic steps. FIG. 4A: Step 1: Characterization of the transcriptional system: Transcriptional components are extracted through an unsupervised “clustering meta-analysis” (FIG. 1). The genes that form each module (designated by a unique ID, e.g. M1.1) possess a consistent transcriptional behavior across all conditions for a defined experimental system. Transcriptional modules are identified by a two digit ID (e.g. 1.1). A graph represents the expression profile of the genes forming a module across multiple conditions (samples). Each module is in turn functionally characterized (e.g. through the analysis of literature profiles). The result is a collection of biologically meaningful transcriptional determinants. FIG. 4B: Step 2: Study perturbations of the system: Comparisons between study groups are performed independently for each module. This analysis permitted identification of changes in expression levels for different conditions (e.g. comparing samples from patients and healthy controls). The results obtained for each module are represented on a graph. The proportion of genes that meet the significance criteria (class comparison) is indicated in a circle, with red being the proportion of significantly over-expressed genes (right half of the pie chart) and blue the proportion of significantly under-expressed genes (top left quardrant of the pie chart). In this theoretical example ¾ genes (75%) with p<0.05 were represented on the graph. Two of these genes are over-expressed (50%—right half of the pie chart) and one is under-expressed (25%—top left quadrant of the pie chart).

FIGS. 5A-B is an analysis of patient blood leukocyte transcriptional profiles. FIG. 5A) Gene level analysis. The upper panel shows a Statistical comparisons identified differentially expressed transcripts between patients with SLE or acute influenza infection and their respective control (p<0.001, Mann Whitney U test, Benjamini and Hochberg False Discovery Rate: SLE=733 transcripts, FLU=234 transcripts). Clustering analysis grouped genes based on expression patterns and results are represented by a heatmap. FIG. 5B) Module level analysis. For each module, gene expression levels obtained for patients (SLE or FLU) and respective healthy volunteer PBMCs were compared (p<0.05, Mann-Whitney rank test). Pie charts indicate the proportion of genes that were significantly changed. Graphs represent transcriptional profiles of the genes that were significantly changed, with each line showing levels of expression (y-axis) of a single transcript across multiple conditions (samples, x-axis). The expression of each gene is normalized to the median of the measurements obtained across all samples. Results obtained for the 28 PBMC transcriptional modules are displayed on a grid. The coordinates are used to indicate module IDs (e.g. M2.8 is row M2, column 8). Spots indicate the proportion of genes that were significantly changed for each module. The proportion of over-expressed genes are shown within a hexagon, and the proportion of under-expressed genes are shown within a circle. Functional interpretation is indicated on a grid by a gray scale.

FIG. 6 Module maps of transcriptional changes caused by disease. For each module, expression levels measured in PBMCs isolated from patients and their respective healthy control group were compared (Mann Whitney Rank test, p<0.05 between: eighteen patients with SLE and eleven healthy volunteers; sixteen patients with acute influenza infection and ten volunteers; sixteen patients with metastatic melanoma and ten volunteers; and sixteen liver transplant recipients vs. ten volunteers). Spots indicate the proportion of genes that were significantly changed for each module. The proportion of over-expressed genes are shown within a hexagon, and the proportion of under-expressed genes are shown within a circle. Results obtained for the twenty-eight PBMC transcriptional modules are displayed on a grid. The coordinates are used to indicate module IDs (e.g. M2.8 is row M2, column 8).

FIGS. 7A-C Analysis of a third-party dataset. Modular microarray data analysis was carried out for a published PBMC gene expression dataset. The study investigated the effects of exercise on gene expression. Blood samples were obtained for fifteen subjects, pre-exercise (Pre), end-exercise (End), and 60 min into recovery (Re). Transcriptional profiles were generated for five pools of three subjects each. Expression profiles are shown for three transcriptional modules (FIG. 7A: M1.1; FIG. 7B: M1.7; FIG. 7C: 2.1). The expression of each gene is normalized to the median of the measurements obtained across all samples. Keywords extracted from the literature are indicated in green.

FIG. 8 Cross-platform validation. PBMC samples from healthy donors and liver transplant recipient were analyzed on two different microarray platforms: Affymetrix U133A&B GeneChips and Illumina Sentrix Human Ref8 BeadChips. The same pools of total RNA were used to independently prepare biotin-labeled cRNA targets. Results are shown for a set of transcripts shared by the two platforms (Affymetrix: upper panel; Illumina: middle panel). The expression of each gene is normalized to the median of the measurements obtained across all samples. The averaged expression values for all the genes forming each transcriptional module are shown in the bottom panel for both Affymetrix and Illumina platforms.

FIG. 9 includes three graphs that the reproducibility of module-level expression data across microarray platforms. PBMC samples from healthy donors and liver transplant recipient were analyzed on two different microarray platforms: Affymetrix U133A&B GeneChips and Illumina Sentrix Human Ref8 BeadChips. The same source of total RNA was used to independently prepare biotin-labeled cRNA targets. Normalized “Modular expression levels” were obtained for each sample by averaging expression values of the genes forming each module. The modular expression levels derived from data generated by Affymetrix and Illumina platforms were highly comparable: Pearson correlation coefficient R²=0.83, 0.98 and 0.93, for M1.2, M3.1 and M3.2 respectively; p<0.0001).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims. Unless defined otherwise, all technical and scientific terms used herein have the meaning commonly understood by a person skilled in the art to which this invention belongs. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991).

Various biochemical and molecular biology methods are well known in the art. For example, methods of isolation and purification of nucleic acids are described in detail in WO 97/10365, WO 97/27317, Chapter 3 of Laboratory Techniques in Biochemistry and Molecular Biology: Hybridization With Nucleic Acid Probes, Part I. Theory and Nucleic Acid Preparation, (P. Tijssen, ed.) Elsevier, N.Y. (1993); Chapter 3 of Laboratory Techniques in Biochemistry and Molecular Biology: Hybridization With Nucleic Acid Probes, Part 1. Theory and Nucleic Acid Preparation, (P. Tijssen, ed.) Elsevier, N.Y. (1993); and Sambrook et al., Molecular Cloning: A Laboratory Manual, Cold Spring Harbor Press, N.Y., (1989); and Current Protocols in Molecular Biology, (Ausubel, F. M. et al., eds.) John Wiley & Sons, Inc., New York (1987-1999), including supplements such as supplement 46 (April 1999).

BIOINFORMATICS DEFINTIONS

As used herein, an “object” refers to any item or information of interest (generally textual, including noun, verb, adjective, adverb, phrase, sentence, symbol, numeric characters, etc.). Therefore, an object is anything that can form a relationship and anything that can be obtained, identified, and/or searched from a source. “Objects” include, but are not limited to, an entity of interest such as gene, protein, disease, phenotype, mechanism, drug, etc. In some aspects, an object may be data, as further described below.

As used herein, a “relationship” refers to the co-occurrence of objects within the same unit (e.g., a phrase, sentence, two or more lines of text, a paragraph, a section of a webpage, a page, a magazine, paper, book, etc.). It may be text, symbols, numbers and combinations, thereof

As used herein, “meta data content” refers to information as to the organization of text in a data source. Meta data can comprise standard metadata such as Dublin Core metadata or can be collection-specific. Examples of metadata formats include, but are not limited to, Machine Readable Catalog (MARC) records used for library catalogs, Resource Description Format (RDF) and the Extensible Markup Language (XML). Meta objects may be generated manually or through automated information extraction algorithms.

As used herein, an “engine” refers to a program that performs a core or essential function for other programs. For example, an engine may be a central program in an operating system or application program that coordinates the overall operation of other programs. The term “engine” may also refer to a program containing an algorithm that can be changed. For example, a knowledge discovery engine may be designed so that its approach to identifying relationships can be changed to reflect new rules of identifying and ranking relationships.

As used herein, “semantic analysis” refers to the identification of relationships between words that represent similar concepts, e.g., though suffix removal or stemming or by employing a thesaurus. “Statistical analysis” refers to a technique based on counting the number of occurrences of each term (word, word root, word stem, n-gram, phrase, etc.). In collections unrestricted as to subject, the same phrase used in different contexts may represent different concepts. Statistical analysis of phrase co-occurrence can help to resolve word sense ambiguity. “Syntactic analysis” can be used to further decrease ambiguity by part-of-speech analysis. As used herein, one or more of such analyses are referred to more generally as “lexical analysis.” “Artificial intelligence (AI)” refers to methods by which a non-human device, such as a computer, performs tasks that humans would deem noteworthy or “intelligent.” Examples include identifying pictures, understanding spoken words or written text, and solving problems.

As used herein, the term “database” refers to repositories for raw or compiled data, even if various informational facets can be found within the data fields. A database is typically organized so its contents can be accessed, managed, and updated (e.g., the database is dynamic). The term “database” and “source” are also used interchangeably in the present invention, because primary sources of data and information are databases. However, a “source database” or “source data” refers in general to data, e.g., unstructured text and/or structured data, that are input into the system for identifying objects and determining relationships. A source database may or may not be a relational database. However, a system database usually includes a relational database or some equivalent type of database which stores values relating to relationships between objects.

As used herein, a “system database” and “relational database” are used interchangeably and refer to one or more collections of data organized as a set of tables containing data fitted into predefined categories. For example, a database table may comprise one or more categories defined by columns (e.g. attributes), while rows of the database may contain a unique object for the categories defined by the columns. Thus, an object such as the identity of a gene might have columns for its presence, absence and/or level of expression of the gene. A row of a relational database may also be referred to as a “set” and is generally defined by the values of its columns. A “domain” in the context of a relational database is a range of valid values a field such as a column may include.

As used herein, a “domain of knowledge” refers to an area of study over which the system is operative, for example, all biomedical data. It should be pointed out that there is advantage to combining data from several domains, for example, biomedical data and engineering data, for this diverse data can sometimes link things that cannot be put together for a normal person that is only familiar with one area or research/study (one domain). A “distributed database” refers to a database that may be dispersed or replicated among different points in a network.

Terms such “data” and “information” are often used interchangeably, as are “information” and “knowledge.” As used herein, “data” is the most fundamental unit that is an empirical measurement or set of measurements. Data is compiled to contribute to information, but it is fundamentally independent of it. Information, by contrast, is derived from interests, e.g., data (the unit) may be gathered on ethnicity, gender, height, weight and diet for the purpose of finding variables correlated with risk of cardiovascular disease. However, the same data could be used to develop a formula or to create “information” about dietary preferences, i.e., likelihood that certain products in a supermarket have a higher likelihood of selling.

As used herein, “information” refers to a data set that may include numbers, letters, sets of numbers, sets of letters, or conclusions resulting or derived from a set of data. “Data” is then a measurement or statistic and the fundamental unit of information. “Information” may also include other types of data such as words, symbols, text, such as unstructured free text, code, etc. “Knowledge” is loosely defined as a set of information that gives sufficient understanding of a system to model cause and effect. To extend the previous example, information on demographics, gender and prior purchases may be used to develop a regional marketing strategy for food sales while information on nationality could be used by buyers as a guideline for importation of products. It is important to note that there are no strict boundaries between data, information, and knowledge; the three terms are, at times, considered to be equivalent. In general, data comes from examining, information comes from correlating, and knowledge comes from modeling.

As used herein, “a program” or “computer program” refers generally to a syntactic unit that conforms to the rules of a particular programming language and that is composed of declarations and statements or instructions, divisible into, “code segments” needed to solve or execute a certain function, task, or problem. A programming language is generally an artificial language for expressing programs.

As used herein, a “system” or a “computer system” generally refers to one or more computers, peripheral equipment, and software that perform data processing. A “user” or “system operator” in general includes a person, that uses a computer network accessed through a ^{“}user device” (e.g., a computer, a wireless device, etc) for the purpose of data processing and information exchange. A “computer” is generally a functional unit that can perform substantial computations, including numerous arithmetic operations and logic operations without human intervention.

As used herein, “application software” or an “application program” refers generally to software or a program that is specific to the solution of an application problem. An “application problem” is generally a problem submitted by an end user and requiring information processing for its solution.

As used herein, a “natural language” refers to a language whose rules are based on current usage without being specifically prescribed, e.g., English, Spanish or Chinese. As used herein, an “artificial language” refers to a language whose rules are explicitly established prior to its use, e.g., computer-programming languages such as C, C++, Java, BASIC, FORTRAN, or COBOL.

As used herein, “statistical relevance” refers to using one or more of the ranking schemes (O/E ratio, strength, etc.), where a relationship is determined to be statistically relevant if it occurs significantly more frequently than would be expected by random chance.

As used herein, the terms “coordinately regulated genes” or “transcriptional modules” are used interchangeably to refer to grouped, gene expression profiles (e.g., signal values associated with a specific gene sequence) of specific genes. Each transcriptional module correlates two key pieces of data, a literature search portion and actual empirical gene expression value data obtained from a gene microarray. The set of genes that is selected into a transcriptional modules is based on the analysis of gene expression data (module extraction algorithm described above). Additional steps are taught by Chaussabel, D. & Sher, A. Mining microarray expression data by literature profiling. Genome Biol 3, RESEARCH0055 (2002), (http://genomebiology.com/2002/3/10/research/0055) relevant portions incorporated herein by reference and expression data obtained from a disease or condition of interest, e.g., Systemic Lupus erythematosus, arthritis, lymphoma, carcinoma, melanoma, acute infection, autoimmune disorders, autoinflammatory disorders, etc.).

The Table below lists examples of keywords that were used to develop the literature search portion or contribution to the transcription modules. The skilled artisan will recognize that other terms may easily be selected for other conditions, e.g., specific cancers, specific infectious disease, transplantation, etc. For example, genes and signals for those genes associated with T cell activation are described hereinbelow as Module ID “M 2.8” in which certain keywords (e.g., Lymphoma, T-cell, CD4, CD8, TCR, Thymus, Lymphoid, IL2) were used to identify key T-cell associated genes, e.g., T-cell surface markers (CD5, CD6, CD7, CD26, CD28, CD96); molecules expressed by lymphoid lineage cells (lymphotoxin beta, IL2-inducible T-cell kinase, TCF7; and T-cell differentiation protein mal, GATA3, STAT5B). Next, the complete module is developed by correlating data from a patient population for these genes (regardless of platform, presence/absence and/or up or downregulation) to generate the transcriptional module. In some cases, the gene profile does not match (at this time) any particular clustering of genes for these disease conditions and data, however, certain physiological pathways (e.g., cAMP signaling, zinc-finger proteins, cell surface markers, etc.) are found within the “Underdetermined” modules. In fact, the gene expression data set may be used to extract genes that have coordinated expression prior to matching to the keyword search, i.e., either data set may be correlated prior to cross-referencing with the second data set.

TABLE 1
Examples of Transcriptional Modules

| Example | | |
|---|---|---|
| Module | Example Keyword | |
| I.D. | selection | Gene Profile Assessment |
| M 1.1 | Ig, Immunoglobulon, Bone, | Plasma cells: Includes genes encoding for Immunoglobulin |
| | Marrow, PreB, IgM, Mu. | chains (e.g. IGHM, IGJ, IGLL1, IGKC, IGHD) and the |
| | | plasma cell marker CD38. |
| M 1.2 | Platelet, Adhesion, | Platelets: Includes genes encoding for platelet glycoproteins |
| | Aggregation, Endothelial, | (ITGA2B, ITGB3, GP6, GP1A/B), and platelet-derived |
| | Vascular | immune mediators such as PPPB (pro-platelet basic protein) |
| | | and PF4 (platelet factor 4). |
| M 1.3 | Immunoreceptor, BCR, B- | B-cells: Includes genes encoding for B-cell surface markers |
| | cell, IgG | (CD72, CD79A/B, CD19, CD22) and other B-cell |
| | | associated molecules: Early B-cell factor (EBF), B-cell |
| | | linker (BLNK) and B lymphoid tyrosine kinase (BLK). |
| M 1.4 | Replication, Repression, | Undetermined. This set includes regulators and targets of |
| | Repair, CREB, Lymphoid, | cAMP signaling pathway (JUND, ATF4, CREM, PDE4, |
| | TNF-alpha | NR4A2, VIL2), as well as repressors of TNF-alpha |
| | | mediated NF-KB activation (CYLD, ASK, TNFAIP3). |
| M 1.5 | Monocytes, Dendritic, | Myeloid lineage: Includes molecules expressed by cells of |
| | MHC, Costimulatory, | the myeloid lineage (CD86, CD163, FCGR2A), some of |
| | TLR4, MYD88 | which being involved in pathogen recognition (CD14, |
| | | TLR2, MYD88). This set also includes TNF family |
| | | members (TNFR2, BAFF). |
| M 1.6 | Zinc, Finger, P53, RAS | Undetermined. This set includes genes encoding for |
| | | signaling molecules, e.g., the zinc finger containing inhibitor |
| | | of activated STAT (PIAS1 and PIAS2), or the nuclear factor |
| | | of activated T-cells NFATC3. |
| M 1.7 | Ribosome, Translational, | MHC/Ribosomal proteins: Almost exclusively formed by |
| | 40S, 60S, HLA | genes encoding MHC class I molecules (HLA-A,B,C,G,E) + |
| | | Beta 2-microglobulin (B2M) or Ribosomal proteins (RPLs, |
| | | RPSs). |
| M 1.8 | Metabolism, Biosyntheses, | Undetermined. Includes genes encoding metabolic enzymes |
| | Replication, Helicase | (GLS, NSF1, NAT1) and factors involved in DNA |
| | | replication (PURA, TERF2, EIF2S1). |
| M 2.1 | NK, Killer, Cytolytic, | Cytotoxic cells: Includes cytotoxic T-cells and NK-cells |
| | CD8, Cell-mediated, T- | surface markers (CD8A, CD2, CD160, NKG7, KLRs), |
| | cell, CTL, IFN-g | cytolytic molecules (granzyme, perforin, granulysin), |
| | | chemokines (CCL5, XCL1) and CTL/NK-cell associated |
| | | molecules (CTSW). |
| M 2.2 | Granulocytes, Neutrophils, | Neutrophils: This set includes innate molecules that are |
| | Defense, Myeloid, Marrow | found in neutrophil granules (Lactotransferrin: LTF, |
| | | defensin: DEAF1, Bacterial Permeability Increasing protein: |
| | | BPI, Cathelicidin antimicrobial protein: CAMP). |
| M 2.3 | Erythrocytes, Red, | Erythrocytes: Includes hemoglobin genes (HGBs) and other |
| | Anemia, Globin, | erythrocyte-associated genes (erythrocytic alkirin: ANK1, |
| | Hemoglobin | Glycophorin C: GYPC, hydroxymethylbilane synthase: |
| | | HMBS, erythroid associated factor: ERAF). |
| M 2.4 | Ribonucleoprotein, 60S, | Ribosomal proteins: Including genes encoding ribosomal |
| | nucleolus, Assembly, | proteins (RPLs, RPSs), Eukaryotic Translation Elongation |
| | Elongation | factor family members (EEFs) and Nucleolar proteins |
| | | (NPM1, NOAL2, NAP1L1). |
| M 2.5 | Adenoma, Interstitial, | Undetermined. This module includes genes encoding |
| | Mesenchyme, Dendrite, | immune-related (CD40, CD80, CXCL12, IFNA5, IL4R) as |
| | Motor | well as cytoskeleton-related molecules (Myosin, Dedicator |
| | | of Cytokenesis, Syndecan 2, Plexin C1, Distrobrevin). |
| M 2.6 | Granulocytes, Monocytes, | Myeloid lineage: Related to M 1.5. Includes genes expressed |
| | Myeloid, ERK, Necrosis | in myeloid lineage cells (IGTB2/CD18, Lymphotoxin beta |
| | | receptor, Myeloid related proteins 8/14 Formyl peptide |
| | | receptor 1), such as Monocytes and Neutrophils: |
| M 2.7 | No keywords extracted. | Undetermined. This module is largely composed of |
| | | transcripts with no known function. Only 20 genes |
| | | associated with literature, including a member of the |
| | | chemokine-like factor superfamily (CKLFSF8). |
| M 2.8 | Lymphoma, T-cell, CD4, | T-cells: Includes T-cell surface markers (CD5, CD6, CD7, |
| | CD8, TCR, Thymus, | CD26, CD28, CD96) and molecules expressed by lymphoid |
| | Lymphoid, IL2 | lineage cells (lymphotoxin beta, IL2-inducible T-cell kinase, |
| | | TCF7, T-cell differentiation protein mal, GATA3, |
| | | STAT5B). |
| M 2.9 | ERK, Transactivation, | Undetermined. Includes genes encoding molecules that |
| | Cytoskeletal, MAPK, JNK | associate to the cytoskeleton (Actin related protein 2/3, |
| | | MAPK1, MAP3K1, RAB5A). Also present are T-cell |
| | | expressed genes (FAS, ITGA4/CD49D, ZNF1A1). |
| M 2.10 | Myeloid, Macrophage, | Undetermined. Includes genes encoding for Immune-related |
| | Dendritic, Inflammatory, | cell surface molecules (CD36, CD86, LILRB), cytokines |
| | Interleukin | (IL15) and molecules involved in signaling pathways (FYB, |
| | | TICAM2-Toll-like receptor pathway). |
| M 2.11 | Replication, Repress, RAS | Undetermined. Includes kinases (UHMK1, CSNK1G1, |
| | Autophosphorylation, | CDK6, WNK1, TAOK1, CALM2, PRKCI, ITPKB, SRPK2, |
| | Oncogenic | STK17B, DYRK2, PIK3R1, STK4, CLK4, PKN2) and RAS |
| | | family members (G3BP, RAB14, RASA2, RAP2A, KRAS). |
| M 3.1 | ISRE, Influenza, Antiviral, | Interferon-inducible: This set includes interferon-inducible |
| | IFN-gamma, IFN-alpha, | genes: antiviral molecules (OAS1/2/3/L, GBP1, G1P2, |
| | Interferon | EIF2AK2/PKR, MX1, PML), chemokines (CXCL10/IP-10), |
| | | signaling molecules (STAT1, STAt2, IRF7, ISGF3G). |
| M 3.2 | TGF-beta, TNF, | Inflammation I: Includes genes encoding molecules |
| | Inflammatory, Apoptotic, | involved in inflammatory processes (e.g., IL8, ICAM1, |
| | Lipopolysaccharide | C5R1, CD44, PLAUR, IL1A, CXCL16), and regulators of |
| | | apoptosis (MCL1, FOXO3A, RARA, BCL3/6/2A1, |
| | | GADD45B). |
| M 3.3 | Granulocyte, | Inflammation II: Includes molecules inducing or inducible |
| | Inflammatory, Defense, | by Granulocyte-Macrophage CSF (SPI1, IL18, ALOX5, |
| | Oxidize, Lysosomal | ANPEP), as well as lysosomal enzymes (PPT1, CTSB/S, |
| | | CES1, NEU1, ASAH1, LAMP2, CAST). |
| M 3.4 | No keyword extracted | Undetermined. Includes protein phosphates (PPP1R12A, |
| | | PTPRC, PPP1CB, PPM1B) and phosphoinositide 3-kinase |
| | | (PI3K) family members (PIK3CA, PIK32A, PIP5K3). |
| M 3.5 | No keyword extracted | Undetermined. Composed of only a small number of |
| | | transcripts. Includes hemoglobin genes (HBA1, HBA2, |
| | | HBB). |
| M 3.6 | Complement, Host, | Undetermined. Large set that includes T-cell surface |
| | Oxidative, Cytoskeletal, T- | markers (CD101, CD102, CD103) as well as molecules |
| | cell | ubiquitously expressed among blood leukocytes (CXRCR1: |
| | | fraktalkine receptor, CD47, P-selectin ligand). |
| M 3.7 | Spliceosome, Methylation, | Undetermined. Includes genes encoding proteasome |
| | Ubiquitin, Beta-catenin | subunits (PSMA2/5, PSMB5/8); ubiquitin protein ligases |
| | | HIP2, STUB1, as well as components of ubiqutin ligase |
| | | complexes (SUGT1). |
| M 3.8 | CDC, TCR, CREB, | Undetermined. Includes genes encoding for several |
| | Glycosylase | enzymes: aminomethyltransferase, arginyltransferase, |
| | | asparagines synthetase, diacylglycerol kinase, inositol |
| | | phosphatases, methyltransferases, helicases . . . |
| M 3.9 | Chromatin, Checkpoint, | Undetermined. Includes genes encoding for protein kinases |
| | Replication, | (PRKPIR, PRKDC, PRKCI) and phosphatases (e.g., |
| | Transactivation | PTPLB, PPP1R8/2CB). Also includes RAS oncogene family |
| | | members and the NK cell receptor 2B4 (CD244). |

BIOLOGICAL DEFINTIONS

As used herein, the term “array” refers to a solid support or substrate with one or more peptides or nucleic acid probes attached to the support. Arrays typically have one or more different nucleic acid or peptide probes that are coupled to a surface of a substrate in different, known locations. These arrays, also described as “microarrays” or “gene-chips” that may have 10,000; 20,000, 30,000; or 40,000 different identifiable genes based on the known genome, e.g., the human genome. These pan-arrays are used to detect the entire “transcriptome” or transcriptional pool of genes that are expressed or found in a sample, e.g., nucleic acids that are expressed as RNA, mRNA and the like that may be subjected to RT and/or RT-PCR to made a complementary set of DNA replicons. Arrays may be produced using mechanical synthesis methods, light directed synthesis methods and the like that incorporate a combination of non-lithographic and/or photolithographic methods and solid phase synthesis methods.

Various techniques for the synthesis of these nucleic acid arrays have been described, e.g., fabricated on a surface of virtually any shape or even a multiplicity of surfaces. Arrays may be peptides or nucleic acids on beads, gels, polymeric surfaces, fibers such as fiber optics, glass or any other appropriate substrate. Arrays may be packaged in such a manner as to allow for diagnostics or other manipulation of an all inclusive device, see for example, U.S. Pat. No. 6,955,788, relevant portions incorporated herein by reference.

As used herein, the term “disease” refers to a physiological state of an organism with any abnormal biological state of a cell. Disease includes, but is not limited to, an interruption, cessation or disorder of cells, tissues, body functions, systems or organs that may be inherent, inherited, caused by an infection, caused by abnormal cell function, abnormal cell division and the like. A disease that leads to a “disease state” is generally detrimental to the biological system, that is, the host of the disease. With respect to the present invention, any biological state, such as an infection (e.g., viral, bacterial, fungal, helminthic, etc.), inflammation, autoinflammation, autoimmunity, anaphylaxis, allergies, premalignancy, malignancy, surgical, transplantation, physiological, and the like that is associated with a disease or disorder is considered to be a disease state. A pathological state is generally the equivalent of a disease state.

Disease states may also be categorized into different levels of disease state. As used herein, the level of a disease or disease state is an arbitrary measure reflecting the progression of a disease or disease state as well as the physiological response upon, during and after treatment. Generally, a disease or disease state will progress through levels or stages, wherein the affects of the disease become increasingly severe. The level of a disease state may be impacted by the physiological state of cells in the sample.

As used herein, the terms “therapy” or “therapeutic regimen” refer to those medical steps taken to alleviate or alter a disease state, e.g., a course of treatment intended to reduce or eliminate the affects or symptoms of a disease using pharmacological, surgical, dietary and/or other techniques. A therapeutic regimen may include a prescribed dosage of one or more drugs or surgery. Therapies will most often be beneficial and reduce the disease state but in many instances the effect of a therapy will have non-desirable or side-effects. The effect of therapy will also be impacted by the physiological state of the host, e.g., age, gender, genetics, weight, other disease conditions, etc.

As used herein, the term “pharmacological state” or “pharmacological status” refers to those samples that will be, are and/or were treated with one or more drugs, surgery and the like that may affect the pharmacological state of one or more nucleic acids in a sample, e.g., newly transcribed, stabilized and/or destabilized as a result of the pharmacological intervention. The pharmacological state of a sample relates to changes in the biological status before, during and/or after drug treatment and may serve a diagnostic or prognostic function, as taught herein. Some changes following drug treatment or surgery may be relevant to the disease state and/or may be unrelated side-effects of the therapy. Changes in the pharmacological state are the likely results of the duration of therapy, types and doses of drugs prescribed, degree of compliance with a given course of therapy, and/or un-prescribed drugs ingested.

As used herein, the term “biological state” refers to the state of the transcriptome (that is the entire collection of RNA transcripts) of the cellular sample isolated and purified for the analysis of changes in expression. The biological state reflects the physiological state of the cells in the sample by measuring the abundance and/or activity of cellular constituents, characterizing according to morphological phenotype or a combination of the methods for the detection of transcripts.

As used herein, the term “expression profile” refers to the relative abundance of RNA, DNA or protein abundances or activity levels. The expression profile can be a measurement for example of the transcriptional state or the translational state by any number of methods and using any of a number of gene-chips, gene arrays, beads, multiplex PCR, quantitiative PCR, run-on assays, Northern blot analysis, Western blot analysis, protein expression, fluorescence activated cell sorting (FACS), enzyme linked immunosorbent assays (ELISA), chemiluminescence studies, enzymatic assays, proliferation studies or any other method, apparatus and system for the determination and/or analysis of gene expression that are readily commercially available.

As used herein, the term “transcriptional state” of a sample includes the identities and relative abundances of the RNA species, especially mRNAs present in the sample. The entire transcriptional state of a sample, that is the combination of identity and abundance of RNA, is also referred to herein as the transcriptome. Generally, a substantial fraction of all the relative constituents of the entire set of RNA species in the sample are measured.

As used herein, the term “modular transcriptional vectors” refers to transcriptional expression data that reflects the “proportion of differentially expressed genes.” For example, for each module the proportion of transcripts differentially expressed between at least two groups (e.g. healthy subjects vs patients). This vector is derived from the comparison of two groups of samples. The first analytical step is used for the selection of disease-specific sets of transcripts within each module. Next, there is the “expression level.” The group comparison for a given disease provides the list of differentially expressed transcripts for each module. It was found that different diseases yield different subsets of modular transcripts. With this expression level it is then possible to calculate vectors for each module(s) for a single sample by averaging expression values of disease-specific subsets of genes identified as being differentially expressed. This approach permits the generation of maps of modular expression vectors for a single sample, e.g., those described in the module maps disclosed herein. These vector module maps represent an averaged expression level for each module (instead of a proportion of differentially expressed genes) that can be derived for each sample.

Using the present invention it is possible to identify and distinguish diseases not only at the module-level, but also at the gene-level; i.e., two diseases can have the same vector (identical proportion of differentially expressed transcripts, identical “polarity”), but the gene composition of the vector can still be disease-specific. Gene-level expression provides the distinct advantage of greatly increasing the resolution of the analysis.

Furthermore, the present invention takes advantage of composite transcriptional markers. As used herein, the term “composite transcriptional markers” refers to the average expression values of multiple genes (subsets of modules) as compared to using individual genes as markers (and the composition of these markers can be disease-specific). The composite transcriptional markers approach is unique because the user can develop multivariate microarray scores to assess disease severity in patients with, e.g., SLE, or to derive expression vectors disclosed herein. Most importantly, it has been found that using the composite modular transcriptional markers of the present invention the results found herein are reproducible across microarray platform, thereby providing greater reliability for regulatory approval.

Gene expression monitoring systems for use with the present invention may include customized gene arrays with a limited and/or basic number of genes that are specific and/or customized for the one or more target diseases. Unlike the general, pan-genome arrays that are in customary use, the present invention provides for not only the use of these general pan-arrays for retrospective gene and genome analysis without the need to use a specific platform, but more importantly, it provides for the development of customized arrays that provide an optimal gene set for analysis without the need for the thousands of other, non-relevant genes. One distinct advantage of the optimized arrays and modules of the present invention over the existing art is a reduction in the financial costs (e.g., cost per assay, materials, equipment, time, personnel, training, etc.), and more importantly, the environmental cost of manufacturing pan-arrays where the vast majority of the data is irrelevant. The modules of the present invention allow for the first time the design of simple, custom arrays that provide optimal data with the least number of probes while maximizing the signal to noise ratio. By eliminating the total number of genes for analysis, it is possible to, e.g., eliminate the need to manufacture thousands of expensive platinum masks for photolithography during the manufacture of pan-genetic chips that provide vast amounts of irrelevant data. Using the present invention it is possible to completely avoid the need for microarrays if the limited probe set(s) of the present invention are used with, e.g., digital optical chemistry arrays, ball bead arrays, beads (e.g., Luminex), multiplex PCR, quantitiative PCR, run-on assays, Northern blot analysis, or even, for protein analysis, e.g., Western blot analysis, 2-D and 3-D gel protein expression, MALDI, MALDI-TOF, fluorescence activated cell sorting (FACS) (cell surface or intracellular), enzyme linked immunosorbent assays (ELISA), chemiluminescence studies, enzymatic assays, proliferation studies or any other method, apparatus and system for the determination and/or analysis of gene expression that are readily commercially available.

The “molecular fingerprinting system” of the present invention may be used to facilitate and conduct a comparative analysis of expression in different cells or tissues, different subpopulations of the same cells or tissues, different physiological states of the same cells or tissue, different developmental stages of the same cells or tissue, or different cell populations of the same tissue against other diseases and/or normal cell controls. In some cases, the normal or wild-type expression data may be from samples analyzed at or about the same time or it may be expression data obtained or culled from existing gene array expression databases, e.g., public databases such as the NCBI Gene Expression Omnibus database.

As used herein, the term “differentially expressed” refers to the measurement of a cellular constituent (e.g., nucleic acid, protein, enzymatic activity and the like) that varies in two or more samples, e.g., between a disease sample and a normal sample. The cellular constituent may be on or off (present or absent), upregulated relative to a reference or downregulated relative to the reference. For use with gene-chips or gene-arrays, differential gene expression of nucleic acids, e.g., mRNA or other RNAs (miRNA, siRNA, hnRNA, rRNA, tRNA, etc.) may be used to distinguish between cell types or nucleic acids. Most commonly, the measurement of the transcriptional state of a cell is accomplished by quantitative reverse transcriptase (RT) and/or quantitative reverse transcriptase-polymerase chain reaction (RT-PCR), genomic expression analysis, post-translational analysis, modifications to genomic DNA, translocations, in situ hybridization and the like.

For some disease states it is possible to identify cellular or morphological differences, especially at early levels of the disease state. The present invention avoids the need to identify those specific mutations or one or more genes by looking at modules of genes of the cells themselves or, more importantly, of the cellular RNA expression of genes from immune effector cells that are acting within their regular physiologic context, that is, during immune activation, immune tolerance or even immune anergy. While a genetic mutation may result in a dramatic change in the expression levels of a group of genes, biological systems often compensate for changes by altering the expression of other genes. As a result of these internal compensation responses, many perturbations may have minimal effects on observable phenotypes of the system but profound effects to the composition of cellular constituents. Likewise, the actual copies of a gene transcript may not increase or decrease, however, the longevity or half-life of the transcript may be affected leading to greatly increases protein production. The present invention eliminates the need of detecting the actual message by, in one embodiment, looking at effector cells (e.g., leukocytes, lymphocytes and/or sub-populations thereof) rather than single messages and/or mutations.

The skilled artisan will appreciate readily that samples may be obtained from a variety of sources including, e.g., single cells, a collection of cells, tissue, cell culture and the like. In certain cases, it may even be possible to isolate sufficient RNA from cells found in, e.g., urine, blood, saliva, tissue or biopsy samples and the like. In certain circumstances, enough cells and/or RNA may be obtained from: mucosal secretion, feces, tears, blood plasma, peritoneal fluid, interstitial fluid, intradural, cerebrospinal fluid, sweat or other bodily fluids. The nucleic acid source, e.g., from tissue or cell sources, may include a tissue biopsy sample, one or more sorted cell populations, cell culture, cell clones, transformed cells, biopies or a single cell. The tissue source may include, e.g., brain, liver, heart, kidney, lung, spleen, retina, bone, neural, lymph node, endocrine gland, reproductive organ, blood, nerve, vascular tissue, and olfactory epithelium.

The present invention includes the following basic components, which may be used alone or in combination, namely, one or more data mining algorithms; one or more module-level analytical processes; the characterization of blood leukocyte transcriptional modules; the use of aggregated modular data in multivariate analyses for the molecular diagnostic/prognostic of human diseases; and/or visualization of module-level data and results. Using the present invention it is also possible to develop and analyze composite transcriptional markers, which may be further aggregated into a single multivariate score.

An explosion in data acquisition rates has spurred the development of mining tools and algorithms for the exploitation of microarray data and biomedical knowledge. Approaches aimed at uncovering the modular organization and function of transcriptional systems constitute promising methods for the identification of robust molecular signatures of disease^{14-16, 17}. Indeed, such analyses can transform the perception of large scale transcriptional studies by taking the conceptualization of microarray data past the level of individual genes or lists of genes.

The present inventors have recognized that current microarray-based research is facing significant challenges with the analysis of data that are notoriously “noisy,” that is, data that is difficult to interpret and does not compare well across laboratories and platforms. A widely accepted approach for the analysis of microarray data begins with the identification of subsets of genes differentially expressed between study groups. Next, the users try subsequently to “make sense” out of resulting gene lists using pattern discovery algorithms and existing scientific knowledge.

Rather than deal with the great variability across platforms, the present inventors have developed a strategy that emphasized the selection of biologically relevant genes at an early stage of the analysis. Briefly, the method includes the identification of the transcriptional components characterizing a given biological system for which an improved data mining algorithm was developed to analyze and extract groups of coordinately expressed genes, or transcriptional modules, from large collections of data.

In one example, twenty-eight transcriptional modules regrouping 4742 probe sets were obtained from 239 blood leukocyte transcriptional profiles. Functional convergence among genes forming these modules was demonstrated through literature profiling. The second step consisted of studying perturbations of transcriptional systems on a modular basis. To illustrate this concept, leukocyte transcriptional profiles obtained from healthy volunteers and patients were obtained, compared and analyzed. Further validation of this gene fingerprinting strategy was obtained through the analysis of a published microarray dataset. Remarkably, the modular transcriptional apparatus, system and methods of the present invention using pre-existing data showed a high degree of reproducibility across two commercial microarray platforms.

The present invention includes the implementation of a widely applicable, two-step microarray data mining strategy designed for the modular analysis of transcriptional systems. This novel approach was used to characterize transcriptional signatures of blood leukocytes, which constitutes the most accessible source of clinically relevant information.

As demonstrated herein, it is possible to determine, differential and/or distinguish between two disease based on two vectors even if the vector is identical (+/+) for two diseases—e.g. M1.3=53% down for both SLE and FLU because the composition of each vector can still be used to differentiate them. For example, even though the proportion and polarity of differentially expressed transcripts is identical between the two diseases for M1.3, the gene composition can still be disease-specific. The combination of gene-level and module-level analysis considerably increases resolution. Furthermore, it is possible to use 2, 3, 4, 5, 10, 15, 20, 25, 28 or more modules to differentiate diseases.

Material and methods. Processing of blood samples. All blood samples were collected in acid citrate dextrose tubes (BD Vacutainer) and immediately delivered at room temperature to the Baylor Institute for Immunology Research, Dallas, TX, for processing. Peripheral blood mononuclear cells (PBMCs) from 3-4 ml of blood were isolated via Ficoll gradient and immediately lysed in RLT reagent (Qiagen, Valencia, Calif.) with beta-mercaptoethanol (BME) and stored at −80° C. prior to the RNA extraction step.

Microarray analysis. Total RNA was isolated using the RNeasy kit (Qiagen) according to the manufacturer's instructions and RNA integrity was assessed using an Agilent 2100 Bioanalyzer (Agilent, Palo Alto, Calif.).

Affymetrix GeneChips: These microarrays include short oligonucleotide probe sets synthesized in situ on a quartz wafer. Target labeling was performed according to the manufacturer's standard protocol (Affymetrix Inc., Santa Clara, Calif.). Biotinylated cRNA targets were purified and subsequently hybridized to Affymetrix HG-U133A and U133B GeneChips (>44,000 probe sets). Arrays were scanned using an Affymetrix confocal laser scanner. Microarray Suite, Version 5.0 (MAS 5.0; Affymetrix) software was used to assess fluorescent hybridization signals, to normalize signals, and to evaluate signal detection calls. Normalization of signal values per chip was achieved using the MAS 5.0 global method of scaling to the target intensity value of 500 per GeneChip. A gene expression analysis software program, GeneSpring, Version 7.1 (Agilent), was used to perform statistical analysis and hierarchical clustering.

Illumina BeadChips: These microarrays include 50 mer oligonucleotide probes attached to 3 m beads, which are lodged into microwells at the surface of a glass slide. Samples were processed and acquired by Illumina Inc. (San Diego, Calif.) on the basis of a service contract. Targets were prepared using the Illumina RNA amplification kit (Ambion, Austin, Tex.). cRNA targets were hybridized to Sentrix HumanRef8 BeadChips (>25,000 probes), which were scanned on an Illumina BeadStation 500. Illumina's Beadstudio software was used to assess fluorescent hybridization signals.

Literature profiling. The literature profiling algorithm employed in this study has been previously described in detail¹⁸. This approach links genes sharing similar keywords. It uses hierarchical clustering, a popular unsupervised pattern discovery algorithm, to analyze patterns of term occurrence in literature abstracts. Step 1: A gene:literature index identifying pertinent publications for each gene is created. Step 2: Term occurrence frequencies were computed by a text processor. Step 3: Stringent filter criteria are used to select relevant keywords (i.e., eliminate terms with either high or low frequency across all genes and retain the few discerning terms characterized by a pattern of high occurrence for only a few genes). Step 4: Two-way hierarchical clustering groups genes and relevant keywords based on occurrence patterns, providing a visual representation of functional relationships existing among a group of genes.

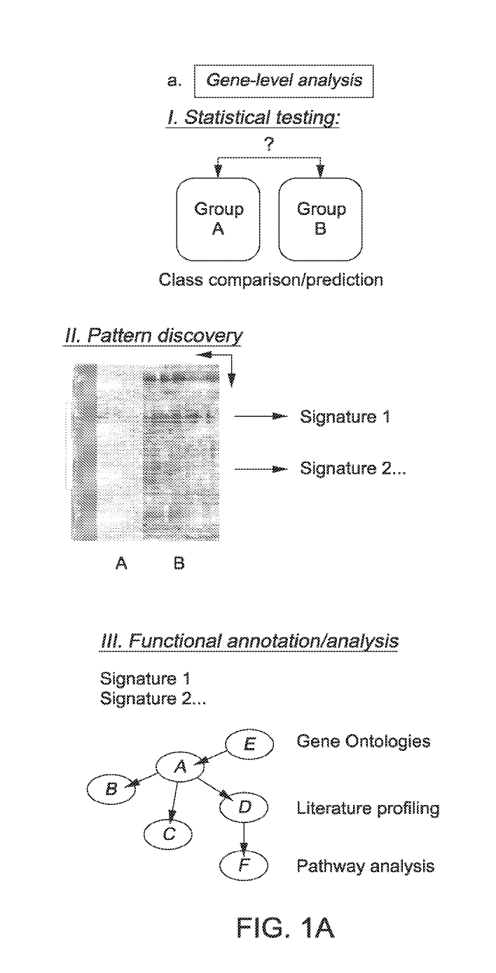

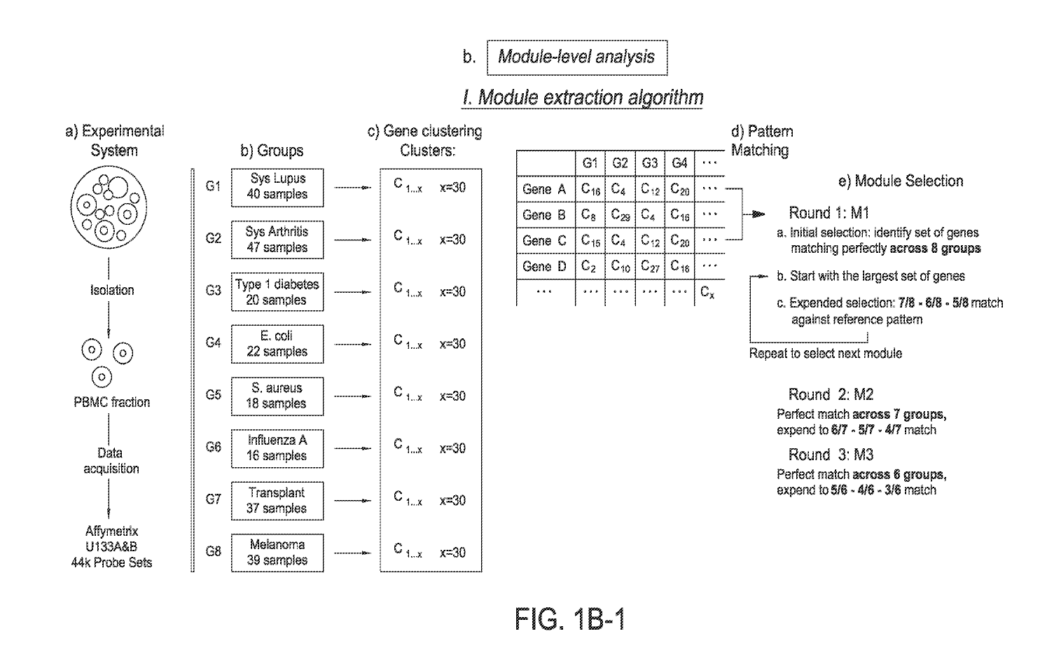

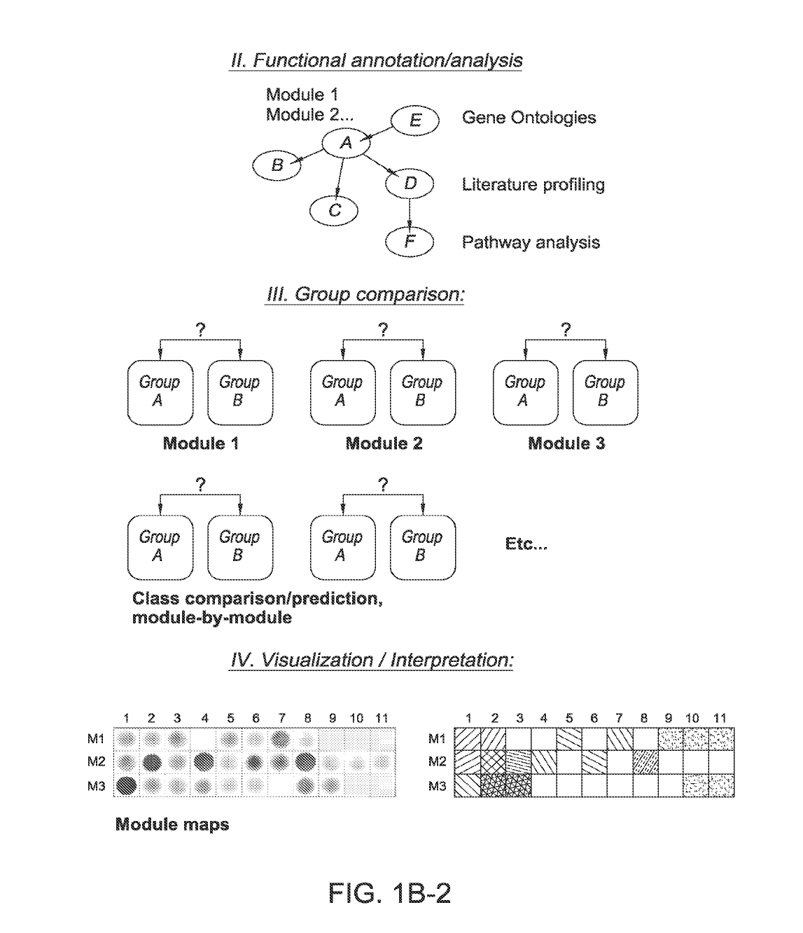

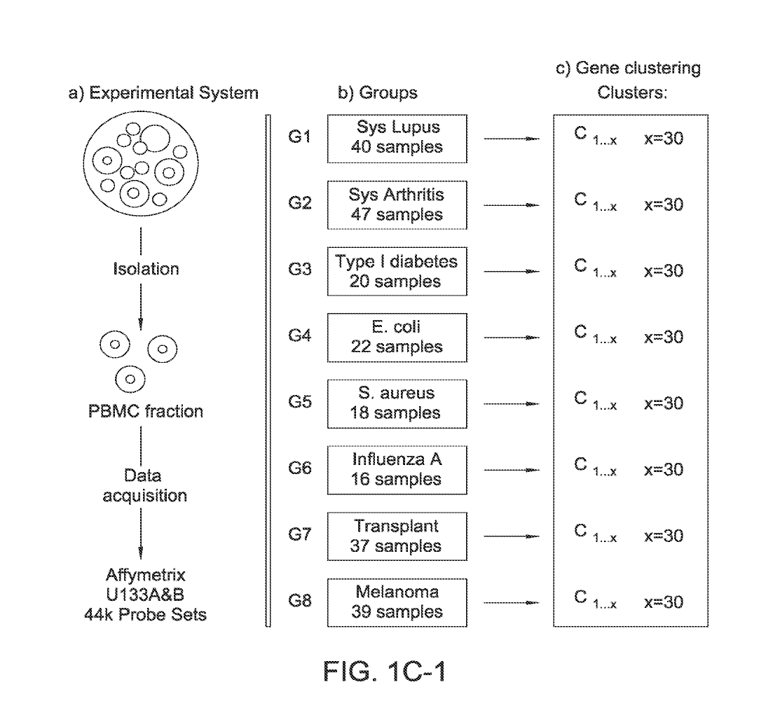

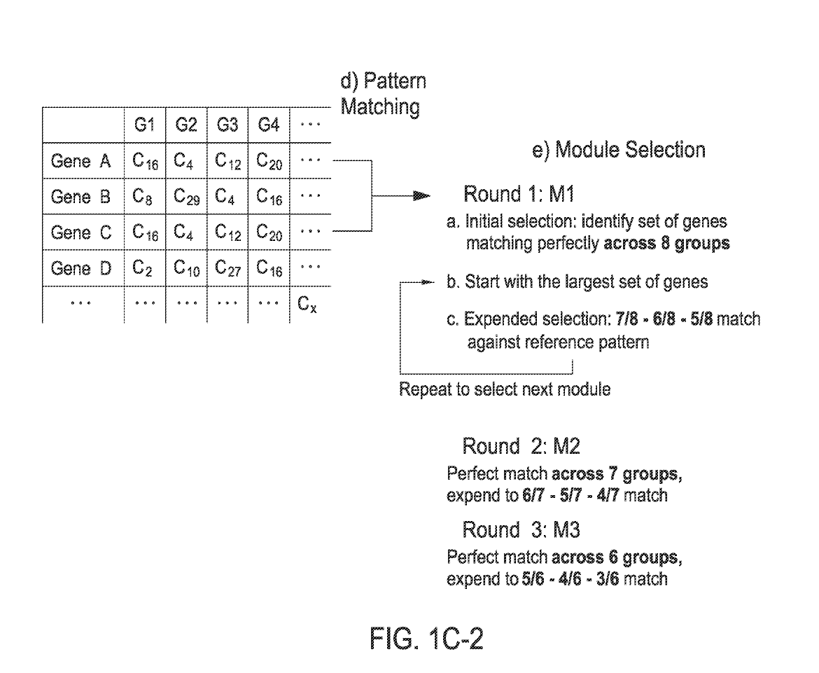

Modular data mining algorithm. First, one or more transcriptional components are identified that permit the characterization of biological systems beyond the level of single genes. Sets of coordinately regulated genes, or transcriptional modules, were extracted using a novel mining algorithm, which was applied to a large set of blood leukocyte microarray profiles (FIG. 1). Gene expression profiles from a total of 239 peripheral blood mononuclear cells (PBMCs) samples were generated using Affymetrix U133A&B GeneChips (>44,000 probe sets). Transcriptional data were obtained for eight experimental groups (systemic juvenile idiopathic arthritis, systemic lupus erythematosus, type I diabetes, liver transplant recipients, melanoma patients, and patients with acute infections: Escherichia coli, Staphylococcus aureus and influenza A). For each group, transcripts with an absent flag call across all conditions were filtered out. The remaining genes were distributed among thirty sets by hierarchical clustering (clusters C1 through C30). The cluster assignment for each gene was recorded in a table and distribution patterns were compared among all the genes. Modules were selected using an iterative process, starting with the largest set of genes that belonged to the same cluster in all study groups (i.e. genes that were found in the same cluster in eight of the eight experimental groups). The selection was then expanded from this core reference pattern to include genes with ⅞, 6/8 and ⅝ matches. The resulting set of genes formed a transcriptional module and was withdrawn from the selection pool. The process was then repeated starting with the second largest group of genes, progressively reducing the level of stringency. This analysis led to the identification of 5348 transcripts that were distributed among twenty-eight modules (a complete list is provided as supplementary material). Each module is assigned a unique identifier indicating the round and order of selection (i.e. M3.1 was the first module identified in the third round of selection).

Modules display distinct “transcriptional behavior”. It is widely assumed that co-expressed genes are functionally linked. This concept of “guilt by association” is particularly compelling in cases where genes follow complex expression patterns across many samples. The present inventors discovered that transcriptional modules form coherent biological units and, therefore, predicted that the co-expression properties identified in our initial dataset would be conserved in an independent set of samples. Data were obtained for PBMCs isolated from the blood of twenty-one healthy volunteers. These samples were not used in the module selection process described above.

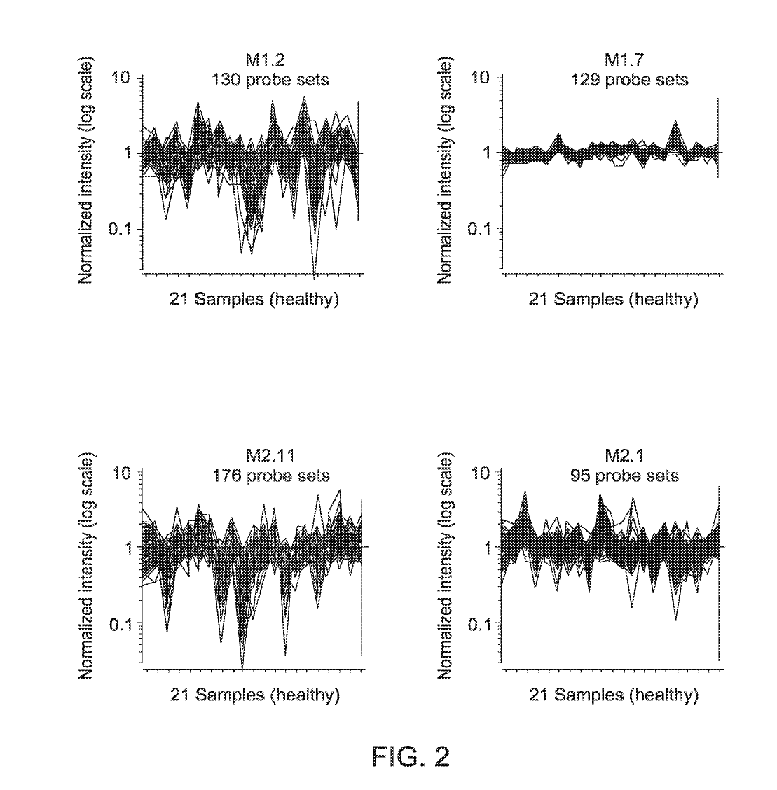

FIG. 2 shows gene expression profiles of four different modules are shown (FIGS. 2: M1.2, M1.7, M2.11 and M2.1). In the graphs of FIG. 2, each line represents the expression level (y-axis) of a single gene across multiple samples (21 samples on the x-axis). Differences in gene expression in this example represent inter-individual variation between “healthy” individuals. It was found that within each module genes display a coherent “transcriptional behavior”. Indeed, the variation in gene expression appeared to be consistent across all the samples (for some samples the expression of all the genes was elevated and formed a peak, while in others levels were low for all the genes which formed a dip). Importantly, inter-individual variations appeared to be module-specific as peaks and dips formed for different samples in M1.2, M2.11 and M2.1. Furthermore, the amplitude of variation was also characteristic of each module, with levels of expression being more variable for M1.2 and M2.11 than M2.1 and especially M1.7. Thus, we find that transcriptional modules constitute independent biological variables.

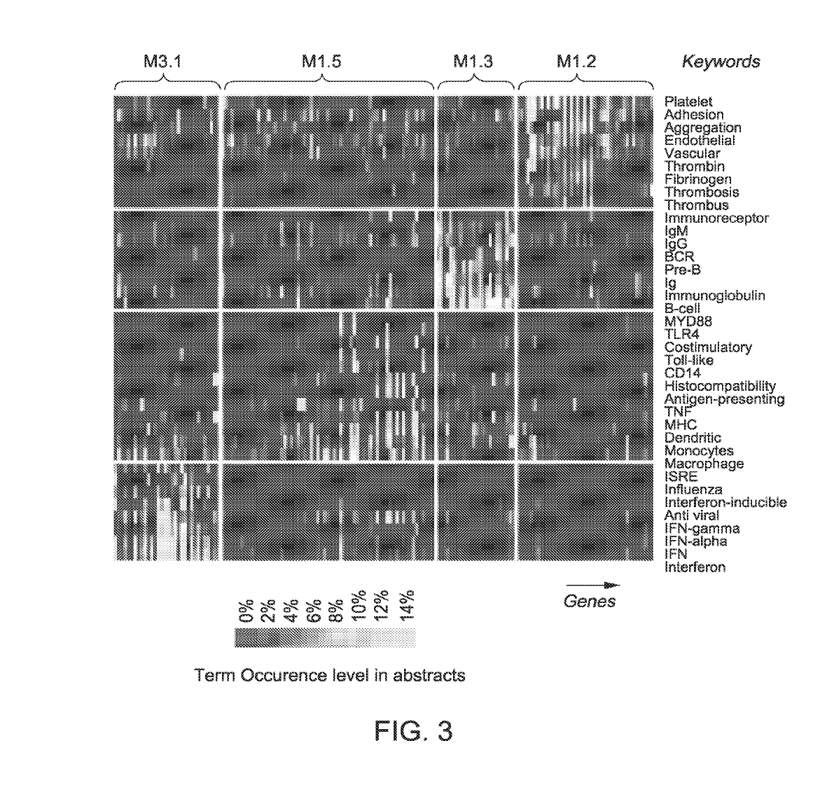

Functional characterization of transcriptional modules. Next, the modules were characterized at a functional level. A text mining approach was employed to extract keywords from the biomedical literature collected for each gene (described in ¹⁸). The distribution of keywords associated to the four modules that were analyzed is clearly distinct (FIG. 3). The following is a list of keywords that may be associated with certain modules.

Keywords highly specific for M1.2 included Platelet, Aggregation or Thrombosis, and were associated with genes such as ITGA2B (Integrin alpha 2b, platelet glycoprotein IIb), PF4 (platelet factor 4), SELP (Selectin P) and GP6 (platelet glycoprotein 6).

Keywords highly specific for M1.3 included B-cell, Immunoglobulin or IgG and were associated with genes such as CD19, CD22, CD72A, BLNK (B cell linker protein), BLK (B lymphoid tyrosine kinase) and PAX5 (paired box gene 5, a B-cell lineage specific activator).

Keywords highly specific for M1.5 included Monocyte, Dendritic, CD14 or Toll-like and were associated with genes such as MYD88 (myeloid differentiation primary response gene 88), CD86, TLR2 (Toll-like receptor 2), LILRB2 (leukocyte immunoglobulin-like receptor B2) and CD163.

Keywords highly specific for M3.1 included Interferon, IFN-alpha, Antiviral, or ISRE and were associated with genes such as STAT1 (signal transducer and activator of transcription 1), CXCL10 (CXC chemokine ligand 10, IP-10), OAS2 (oligoadenylate synthetase 2) and MX2 (myxovirus resistance 2).

This contrasted pattern of term occurrence denotes the remarkable functional coherence of each module. Information extracted from the literature for all the modules that have been identified permit a comprehensive functional characterization of the PBMC system at a transcriptional level. A description of functional associations identified for each of the twenty-eight sample PBMC transcriptional modules is provided in Table 2.

TABLE 2
Complete Functional assessment of 28 transcriptional modules

| Module | Number of | | |
|---|---|---|---|
| I.D. | probe sets | Keyword selection | Assessment |
| M 1.1 | 69 | Ig, Immunoglobulin, | Plasma cells: Includes genes encoding for |
| | | Bone, Marrow, PreB, | Immunoglobulin chains (e.g. IGHM, IGJ, IGLL1, |
| | | IgM, Mu. | IGKC, IGHD) and the plasma cell marker CD38. |
| M 1.2 | 96 | Platelet, Adhesion, | Platelets: Includes genes encoding for platelet |
| | | Aggregation, | glycoproteins (ITGA2B, ITGB3, GP6, GP1A/B), and |
| | | Endothelial, Vascular | platelet-derived immune mediators such as PPPB (pro- |
| | | | platelet basic protein) and PF4 (platelet factor 4). |
| M 1.3 | 47 | Immunoreceptor, | B-cells: Includes genes encoding for B-cell surface |
| | | BCR, B-cell, IgG | markers (CD72, CD79A/B, CD19, CD22) and other |
| | | | B-cell associated molecules: Early B-cell factor (EBF), |
| | | | B-cell linker (BLNK) and B lymphoid tyrosine kinase |
| | | | (BLK). |
| M 1.4 | 87 | Replication, | Undetermined. This set includes regulators and targets |
| | | Repression, Repair, | of cAMP signaling pathway (JUND, ATF4, CREM, |
| | | CREB, Lymphoid, | PDE4, NR4A2, VIL2), as well as repressors of TNF- |
| | | TNF-alpha | alpha mediated NF-KB activation (CYLD, ASK, |
| | | | TNFAIP3). |
| M 1.5 | 130 | Monocytes, | Myeloid lineage: Includes molecules expressed by |
| | | Dendritic, MHC, | cells of the myeloid lineage (CD86, CD163, |
| | | Costimulatory, | FCGR2A), some of which being involved in pathogen |
| | | TLR4, MYD88, | recognition (CD14, TLR2, MYD88). This set also |
| | | | includes TNF family members (TNFR2, BAFF). |
| M 1.6 | 28 | Zinc, Finger, P53, | Undetermined. This set includes genes encoding for |
| | | RAS | signaling molecules, e.g. the zinc finger containing |
| | | | inhibitor of activated STAT (PIAS1 and PIAS2), or |
| | | | the nuclear factor of activated T-cells NFATC3. |
| M 1.7 | 127 | Ribosome, | MHC/Ribosomal proteins: Almost exclusively formed |
| | | Translational, 40S, | by genes encoding MHC class I molecules (HLA- |
| | | 60S, HLA | A,B,C,G,E) + Beta 2-microglobulin (B2M) or |
| | | | Ribosomal proteins (RPLs, RPSs). |
| M 1.8 | 86 | Metabolism, | Undetermined. Includes genes encoding metabolic |
| | | Biosynthesis, | enzymes (GLS, NSF1, NAT1) and factors involved in |
| | | Replication, Helicase | DNA replication (PURA, TERF2, EIF2S1). |
| M 2.1 | 72 | NK, Killer, | Cytotoxic cells: Includes cytotoxic T-cells amd NK- |
| | | Cytolytic, CD8, Cell- | cells surface markers (CD8A, CD2, CD160, NKG7, |
| | | mediated, T-cell, | KLRs), cytolytic molecules (granzyme, perforin, |
| | | CTL, IFN-g | granulysin), chemokines (CCL5, XCL1) and CTL/NK- |
| | | | cell associated molecules (CTSW). |
| M 2.2 | 44 | Granulocytes, | Neutrophils: This set includes innate molecules that |
| | | Neutrophils, | are found in neutrophil granules (Lactotransferrin: |
| | | Defense, Myeloid, | LTF, defensin: DEAF1, Bacterial Permeability |
| | | Marrow | Increasing protein: BPI, Cathelicidin antimicrobial |
| | | | protein: CAMP . . . ). |
| M 2.3 | 94 | Erythrocytes, Red, | Erythrocytes: Includes hemoglobin genes (HGBs) and |
| | | Anemia, Globin, | other erythrocyte-associated genes (erythrocytic |
| | | Hemoglobin | alkirin:ANK1, Glycophorin C: GYPC, |
| | | | hydroxymethylbilane synthase: HMBS, erythroid |
| | | | associated factor: ERAF). |
| M 2.4 | 118 | Ribonucleoprotein, | Ribosomal proteins: Including genes encoding |
| | | 60S, nucleolus, | ribosomal proteins (RPLs, RPSs), Eukaryotic |
| | | Assembly, | Translation Elongation factor family members (EEFs) |
| | | Elongation | and Nucleolar proteins (NPM1, NOAL2, NAP1L1). |
| M 2.5 | 242 | Adenoma, | Undetermined. This module includes genes encoding |
| | | Interstitial, | immune-related (CD40, CD80, CXCL12, IFNA5, |
| | | Mesenchyme, | IL4R) as well as cytoskeleton-related molecules |
| | | Dendrite, Motor | (Myosin, Dedicator of Cytokenesis, Syndecan 2, |
| | | | Plexin C1, Distrobrevin). |
| M 2.6 | 110 | Granulocytes, | Myeloid lineage: Related to M 1.5. Includes genes |
| | | Monocytes, Myeloid, | expressed in myeloid lineage cells (IGTB2/CD18, |
| | | ERK, Necrosis | Lymphotoxin beta receptor, Myeloid related proteins |
| | | | 8/14 Formyl peptide receptor 1), such as Monocytes |
| | | | and Neutrophils: |
| M 2.7 | 43 | No keywords | Undetermined. This module is largely composed of |
| | | extracted. | transcripts with no known function. Only 20 genes |
| | | | associated with literature, including a member of the |
| | | | chemokine-like factor superfamily (CKLFSF8). |
| M 2.8 | 104 | Lymphoma, T-cell, | T-cells: Includes T-cell surface markers (CD5, CD6, |
| | | CD4, CD8, TCR, | CD7, CD26, CD28, CD96) and molecules expressed |
| | | Thymus, Lymphoid, | by lymphoid lineage cells (lymphotoxin beta, IL2- |
| | | IL2 | inducible T-cell kinase, TCF7, T-cell differentiation |
| | | | protein mal, GATA3, STAT5B). |
| M 2.9 | 122 | ERK, | Undetermined. Includes genes encoding molecules |
| | | Transactivation, | that associate to the cytoskeleton (Actin related protein |
| | | Cytoskeletal, MAPK, | 2/3, MAPK1, MAP3K1, RAB5A). Also present are T- |
| | | JNK | cell expressed genes (FAS, ITGA4/CD49D, ZNF1A1). |
| M 2.10 | 44 | Myeloid, | Undetermined. Includes genes encoding for Immune- |
| | | Macrophage, | related cell surface molecules (CD36, CD86, LILRB), |
| | | Dendritic, | cytokines (IL15) and molecules involved in signaling |
| | | Inflammatory, | pathways (FYB, TICAM2-Toll-like receptor |
| | | Interleukin | pathway). |
| M 2.11 | 77 | Replication, Repress, | Undetermined. Includes kinases (UHMK1, CSNK1G1, |
| | | RAS, | CDK6, WNK1, TAOK1, CALM2, PRKCI, ITPKB, |
| | | Autophosphorylation, | SRPK2, STK17B, DYRK2, PIK3R1, STK4, CLK4, |
| | | Oncogenic | PKN2) and RAS family members (G3BP, RAB14, |
| | | | RASA2, RAP2A, KRAS). |
| M 3.1 | 80 | ISRE, Influenza, | Interferon-inducible: This set includes interferon- |
| | | Antiviral, IFN- | inducible genes: antiviral molecules (OAS1/2/3/L, |
| | | gamma, IFN-alpha, | GBP1, G1P2, EIF2AK2/PKR, MX1, PML), |
| | | Interferon | chemokines (CXCL10/IP-10), signaling molecules |
| | | | (STAT1, STAt2, IRF7, ISGF3G). |
| M 3.2 | 230 | TGF-beta, TNF, | Inflammation I: Includes genes encoding molecules |
| | | Inflammatory, | involved in inflammatory processes (e.g. IL8, ICAM1, |
| | | Apoptotic, | C5R1, CD44, PLAUR, IL1A, CXCL16), and |
| | | Lipopolysaccharide | regulators of apoptosis (MCL1, FOXO3A, RARA, |
| | | | BCL3/6/2A1, GADD45B). |
| M 3.3 | 230 | Granulocyte, | Inflammation II: Includes molecules inducing or |
| | | Inflammatory, | inducible by Granulocyte-Macrophage CSF (SPI1, |
| | | Defense, Oxidize, | IL18, ALOX5, ANPEP), as well as lysosomal |
| | | Lysosomal | enzymes (PPT1, CTSB/S, CES1, NEU1, ASAH1, |
| | | | LAMP2, CAST). |
| M 3.4 | 323 | No keyword | Undetermined. Includes protein phosphates |
| | | extracted | (PPP1R12A, PTPRC, PPP1CB, PPM1B) and |
| | | | phosphoinositide 3-kinase (PI3K) family members |
| | | | (PIK3CA, PIK32A, PIP5K3). |
| M 3.5 | 19 | No keyword | Undetermined. Composed of only a small number of |
| | | extracted | transcripts. Includes hemoglobin genes (HBA1, |
| | | | HBA2, HBB). |
| M 3.6 | 233 | Complement, Host, | Undetermined. This very large set includes T-cell |
| | | Oxidative, | surface markers (CD101, CD102, CD103) as well as |
| | | Cytoskeletal, T-cell | molecules ubiquitously expressed among blood |
| | | | leukocytes (CXRCR1: fraktalkine receptor, CD47, P- |
| | | | selectin ligand). |
| M 3.7 | 80 | Spliceosome, | Undetermined. Includes genes encoding proteasome |
| | | Methylation, | subunits (PSMA2/5, PSMB5/8); ubiquitin protein |
| | | Ubiquitin, Beta- | ligases HIP2, STUB1, as well as components of |
| | | catenin | ubiqutin ligase complexes (SUGT1). |
| M 3.8 | 182 | CDC, TCR, CREB, | Undetermined. Includes genes encoding for several |
| | | Glycosylase | enzymes: aminomethyltransferase, arginyltransferase, |
| | | | asparagines synthetase, diacylglycerol kinase, inositol |
| | | | phosphatases, methyltransferases, helicases . . . |
| M 3.9 | 261 | Chromatin, | Undetermined. Includes genes encoding for protein |
| | | Checkpoint, | kinases (PRKPIR, PRKDC, PRKCI) and phosphatases |
| | | Replication, | (e.g. PTPLB, PPP1R8/2CB). Also includes RAS |
| | | Transactivation | oncogene family members and the NK cell receptor |
| | | | 2B4 (CD244). |

Module-based microarray data mining strategy. Results from “traditional” microarray analyses are notoriously noisy and difficult to interpret. A widely accepted approach for microarray data analyses includes three basic steps: 1) Use of a statistical test to select genes differentially expressed between study groups; 2) Apply pattern discovery algorithms to identify signatures among the resulting gene lists; and 3) Interpret the data using knowledge derived from the literature or ontology databases.

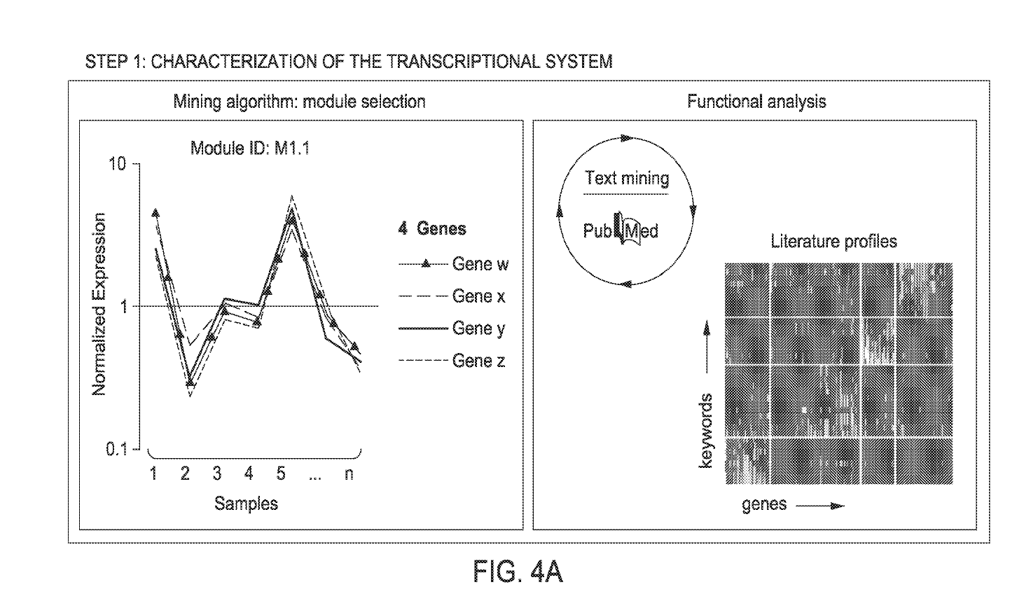

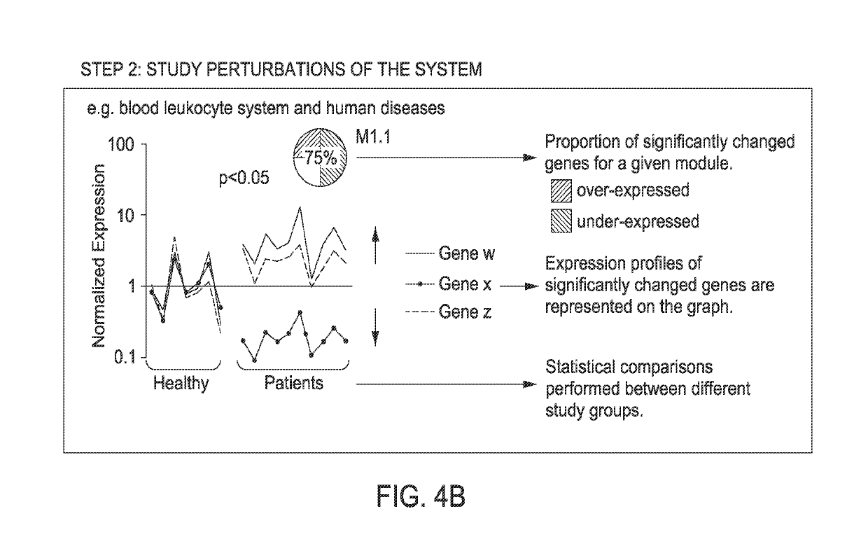

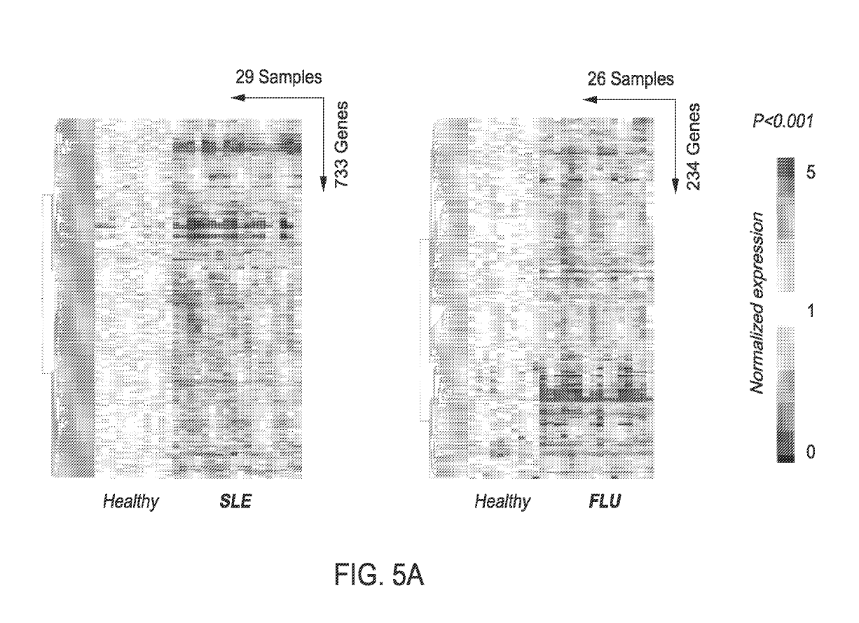

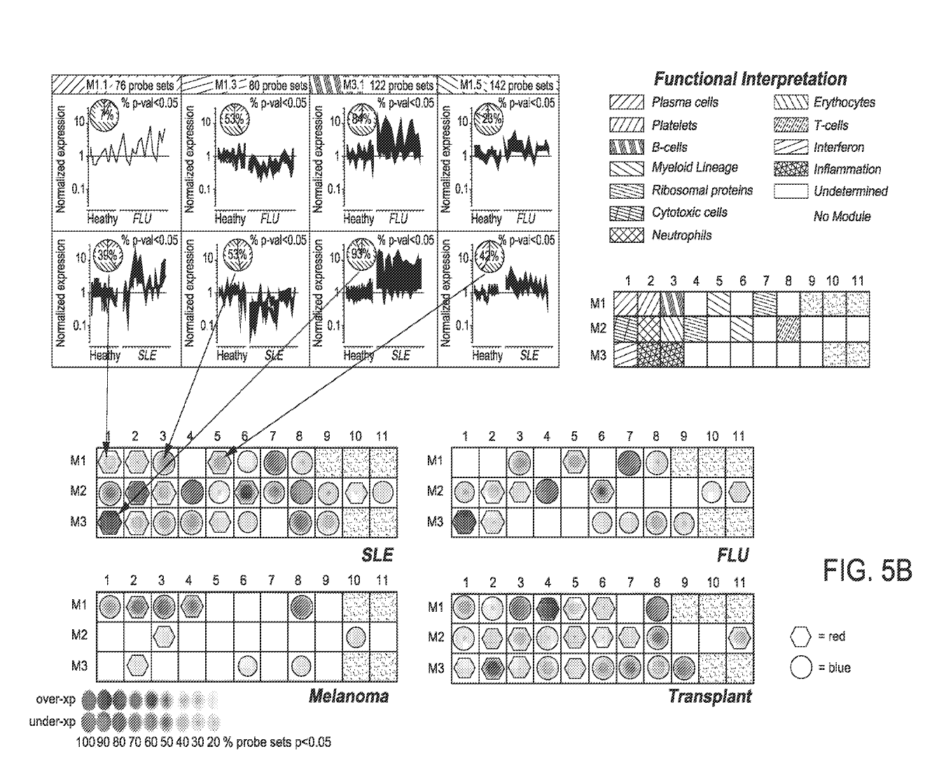

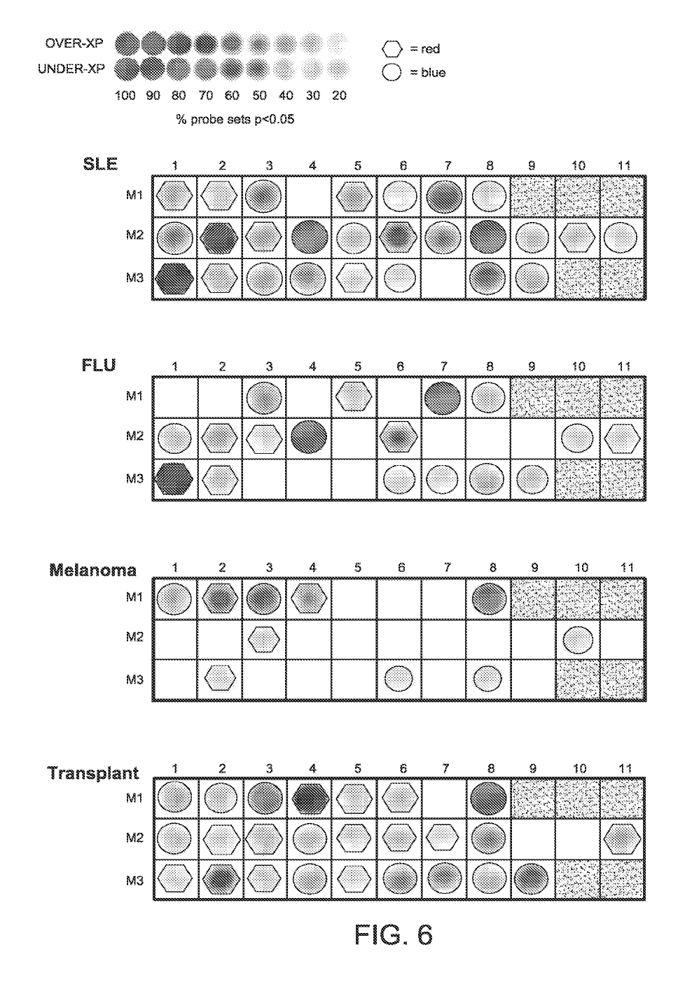

The present invention uses a novel microarray data mining strategy emphasizing the selection of biologically relevant transcripts at an early stage of the analysis. This first step can be carried out using for instance the modular mining algorithm described above in combination with a functional mining tool used for in-depth characterization of each transcriptional module (FIG. 4: top panel, Step 1). The analysis does not take into consideration differences in gene expression levels between groups. Rather, the present invention focuses instead on complex gene expression patterns that arise due to biological variations (e.g. inter-individual variations among a patient population). After defining the transcriptional components associated to a given biological system the second step of the analysis includes the analysis of changes in gene expression through the comparison of different study groups (FIG. 4: bottom panel, Step 2). Group comparison analyses are carried out independently for each module. Changes at the module level are expressed as the proportion of genes that meet the significance criteria (represented by a pie chart in FIG. 5 or a spot in FIG. 6). Notably, carrying out comparisons at the modular level permits to avoid the noise generated when thousands of tests are performed on “random” collections of genes.

Perturbation of modular PBMC transcriptional profiles in human diseases. To illustrate the second step of the microarray data mining strategy described above (FIG. 4), gene expression data for PBMC samples obtained from two pediatric patient populations composed of eighteen children with systemic lupus erythematosus (SLE) and sixteen children with acute influenza A infection was obtained, compared and analyzed. Each patient cohort was matched to its respective control group (healthy volunteers: eleven and ten donors were matched to the SLE and influenza groups, respectively). Following the analytical scheme depicted in FIG. 4, a statistical group comparisons between patient and healthy groups for each individual module and measured the proportion of genes significantly changed in each module (FIG. 5) was performed. The statistical group comparison approach allows the user to focus the analysis on well defined groups of genes that contain minimal amounts of noise and carry identifiable biological meaning. A key to the graphical representation of these results is provided in FIG. 4.

The following findings were made: (1) that a large proportion of genes in M3.1 (“interferon-associated”) met the significance level in both Flu and SLE groups (84% and 94%, respectively). This observation confirms earlier work with SLE patients ¹⁹ and identifies the presence of an interferon signature in patients with acute influenza infection. (2) Equivalent proportions of genes in M1.3 (“B-cell-associated”) were significantly changed in both groups (53%), with over 50% overlap between the two lists. This time, genes were consistently under-expressed in patient compared to healthy groups. (3) Modules were also found that differentiate the two diseases. The proportion of genes significantly changed in Module 1.1 reaches 39% in SLE patients and is only 7% in Flu patients, which at a significance level of 0.05 is very close to the proportion of genes that would be expected to be differentially expressed only by chance. Interestingly, this module is almost exclusively composed of genes encoding immunoglobin chains and has been associated with Plasma cells: However, this module is clearly distinct from the B-cell associated module (M1.3), both in terms of gene expression level and pattern (not shown). (4) As illustrated by module M1.5, gene-level analysis of individual modules can be used to further discriminate the two diseases. It is also the case for M1.3, where, despite the absence of differences at the module-level (FIG. 4: 53% under-expressed transcripts), differences between Flu and SLE groups could be identified at the gene-level (only 51% of the under-expressed transcripts in M1.3 were common to the two disease groups). These examples illustrate the use of a modular framework to streamline the analysis and interpretation of microarray results.

Mapping changes in gene expression at the modular level. Data visualization is paramount for the interpretation of complex datasets and we sought to provide a comprehensive graphical illustration of changes that occur at the modular level. Changes in gene expression levels caused by different diseases were represented for the twenty-eight PBMC transcriptional modules (FIG. 6). Each disease group is compared to its respective control group composed of healthy donors who were matched for age and sex (eighteen patients with SLE, sixteen with acute influenza infection, sixteen with metastatic melanoma and sixteen liver transplant recipients receiving immunosuppressive drug treatment were compared to control groups composed of ten to eleven healthy subjects). Module-level data were represented graphically by spots aligned on a grid, with each position corresponding to a different module (See Table 1 for functional annotations on each of the modules).

The spot intensity indicates the proportion of genes significantly changed for each module. The spot color indicates the polarity of the change (red: proportion of over-expressed genes, blue: proportion of under-expressed genes; modules containing a significant proportion of both over- and under-expressed genes would be purple-though none were observed). This representation permits a rapid assessment of perturbations of the PBMC transcriptional system. Such “module maps” were generated for each disease. When comparing the four maps, we found that diseases were characterized by a unique modular combination. Indeed, results for M1.1 and M1.2 alone sufficed to distinguish all four diseases (M1.1/M1.2: SLE =+/+; FLU=0/0; Melanoma=−/+; transplant=−/−). A number of genes in M3.2 (“inflammation”) were over-expressed in all diseases (particularly so in the transplant group), while genes in M3.1 (interferon) were over-expressed in patients with SLE, influenza infection and, to some extent, transplant recipients. “Ribosomal protein” module genes (M1.7 and M2.4) were under-expressed in both SLE and Flu groups. The level of expression of these genes was recently found to be inversely correlated to disease activity in SLE patients (Bennett et al., submitted). M2.8 includes T-cell transcripts which are under-expressed in lymphopenic SLE patients and transplant recipients treated with immunosuppressive drugs targeting T-cells:

Interestingly, differentially expressed genes in each module were predominantly either under-expressed or over-expressed (FIG. 5 and FIG. 6). Yet, modules were purely selected on the basis of similarities in gene expression profiles, not changes in expression levels between groups. The fact that changes in gene expression appear highly polarized within each module denotes the functional relevance of modular data. Thus, the present invention enables disease fingerprinting by a modular analysis of patient blood leukocyte transcriptional profiles.

Validation of PBMC modules in a published dataset. Next, the validity of the PBMC transcriptional modules described above in a “third-party” dataset was tested. The study from Connolly, et al., who investigated the effects of exercise on gene expression in human PBMCs²⁰ was tested.

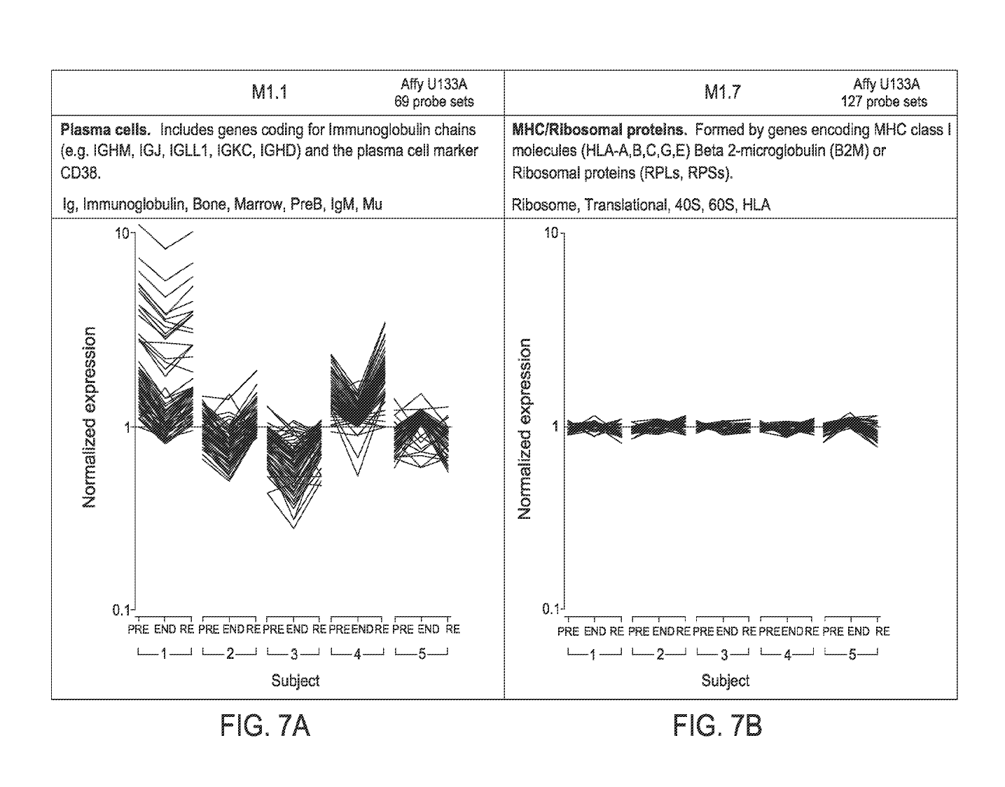

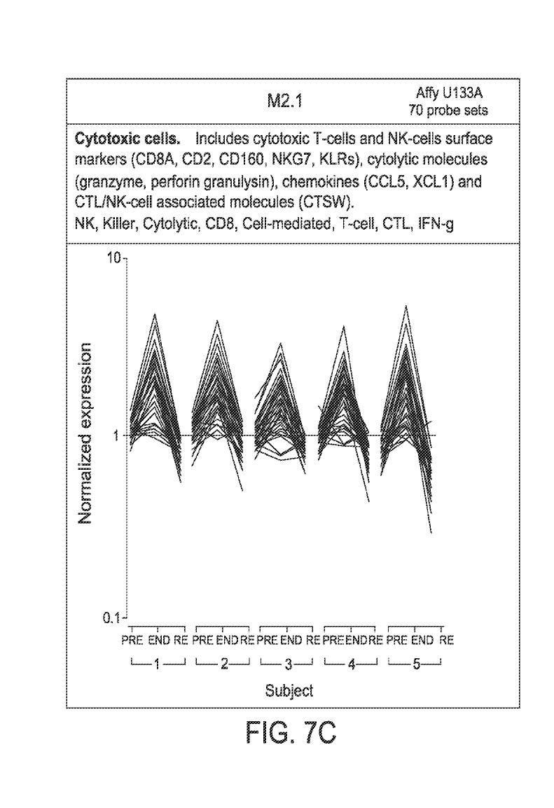

Briefly, samples were obtained from fifteen healthy men prior to and immediately after performing thirty minutes of constant work rate cycle ergometry and one hour after the end of the exercise. Transcriptional profiles were generated for five RNA pools of three subjects each, using Affymetrix U133A gene chips. Raw expression data was downloaded from the NCBI Gene Expression Omnibus website ²¹ and analyzed changes in gene expression on a module-by-module basis. FIG. 7 shows transcriptional profiles of modules M1.1 (“plasma cells”), M1.7 (“ribosomal proteins”) and M2.1 (“cytotoxic cells”). Gene transcriptional behavior for each of these modules was clearly distinct. Interestingly, differences were found between subject pools (M1.1), experimental conditions (M2.1), or no differences (M1.7). These data clearly indicate an increase in expression of cytotoxic cell associated genes (M2.1) immediately after exercise, followed by a decrease to levels comparable to baseline after recuperation. This finding is consistent with the elevation in circulating natural killer cells observed after exercise in sedentary subjects^{22,23}. Some of the genes included in M2.1 were listed by Connolly et al. under the category “inflammatory response”, but the author did not make the link with a possible change in cellular composition. Very few genes belonging to “inflammatory” modules (M3.2, M3.3) were found to be changed after exercise, despite the fact that levels of expression of the genes composing these modules are increased in a wide range of diseases (Chaussabel et al., submitted). Interestingly, however, immunosuppressive molecules specifically over-expressed in patients with stage IV melanoma and transplant patients (Chaussabel et al., submitted) were found to be transiently increased after exercise (not shown, M1.4; e.g. TCF8, CREM, RGS1, TNFAIP3).

Taken together the results from this analysis demonstrate the validity of the proposed modular mining strategy in the context of data generated by an independent group of investigators. Using the present invention, it was found that modular transcriptional data are reproducible across microarray platforms.

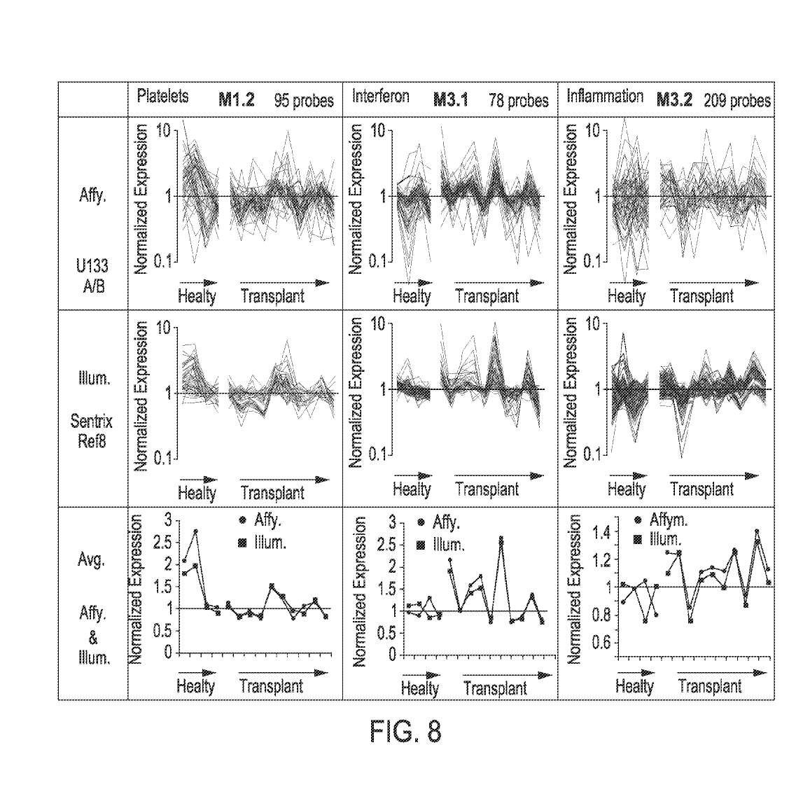

First, modular transcriptional profiles obtained using two commercial microarray platforms were compared. PBMCs were isolated from fourteen samples donated by four healthy volunteers and ten liver transplant recipients. Starting from the same source of total RNA, targets were generated independently and analyzed using Affymetrix U133 GeneChips (at the Baylor Institute for Immunology Research) and Illumina Human Ref8 BeadChips (at the Illumina service core). Fundamental differences exist between the two microarray technologies (see Methods for details). Probe IDs provided by each manufacturer were converted into a unique ID (NCBI Entrez gene ID) that was used for matching gene expression profiles. Data obtained for shared sets of genes are shown in FIG. 8 for modules M1.2 (“platelets”), M3.1 (“interferon”) and M3.2 (“inflammation”). Profiles derived from data obtained with Illumina beadchips show a very high level of co-expression among genes within each module. This observation is particularly meaningful since the selection of transcriptional modules was exclusively based on gene expression data generated using Affymetrix GeneChips. Furthermore, averaged gene expression values for each module were highly reproducible across microarray platforms (FIG. 8).

These results demonstrate the robustness of modular transcriptional signatures and clearly indicate that module-level analysis has the potential to address concerns regarding the reproducibility of microarray data generated at different locations and with different platforms.

Microarray gene expression data produce a comprehensive, but disorganized view of biological systems. Challenges faced by microarray-based research are threefold: (1) Noise, (2) data interpretation and (3) reproducibility. As regards noise, the present invention successfully compared tens of thousands of genes, which the prior art methods invariably produce results that include a large proportion of noise²⁴. As regards data interpretation, the present invention overcomes the problem of information overload. Indeed, interpreting microarray data often requires investigators to examine experimental data in the context of existing biomedical knowledge, on a genome-wide scale ¹³. More unsettling is the possibility of generating spurious results through the over-interpretation of noisy data ⁷. Finally, as regards reproducibility it is well documented that a key problem with existing technology is the poor reproducibility of microarray results obtained by different laboratories and across platforms has been disconcerting and remains, to this date, a major concern^{6,7,10-12}.

Mainstream microarray analysis strategies have had limited success in addressing this triad of issues, for several reasons. First of all, because statistical tests are considered as the prerequisite initial step of the analysis. As a consequence, biological considerations come into play only once a list of differentially expressed genes has been generated. Data subsets resulting from the testing of tens of thousands of variables will, however, invariably contain noise and are, therefore, particularly difficult to interpret. The system and method of the present invention takes the cellular and molecular biology of the cells into consideration when determining the features of the modules. In the present invention the first step is to take into account the biology of the system in the very first step of the analysis, thereby selecting sets of functionally-linked genes found to be coordinately expressed across hundreds of samples. Statistical testing is then applied to modular datasets which are considerably enriched in biologically meaningful genes. An additional benefit of this approach is that it transcends gene level analysis by using transcriptional modules as elementary units. Transcriptional modules constitute a framework for the analysis of perturbations that occur in the context of a defined biological system. This modular data format helps streamline the interpretation of microarray studies. It requires, however, the preliminary characterization of each experimental system under a broad range of biological variables, e.g., different experimental conditions, inter-individual variations, and cost or access to biological material can be a limitation.

Interestingly, the data derived from module-level analyses proved to be particularly robust, as indicated by the excellent reproducibility obtained across two commercial microarray platforms. Furthermore, multivariate analysis of PBMC transcriptional modules led to the establishment of a “genomic score,” which provided an accurate assessment of disease severity in patients with systemic lupus erythematosus (Bennett, et al., submitted). The identification of reliable blood leukocyte transcriptional markers constitutes an important step towards the application of microarrays in clinical settings.

Working with samples formed by multiple cell types adds a level of complexity to the analysis of microarray gene expression data. Indeed, differences of gene expression levels can be explained not only by changes in transcriptional activity but also changes in cellular composition. Modular signatures obtained analyzing PBMC samples reflect this fact and permit us to distinguish cellular components (including genes associated to platelets—M1.2 -, erythrocytes—M2.3 or T-cells—M2.8) from components related to activation (including genes associated to interferon—M3.1, inflammation M3.2, or signaling—M2.11). This type of consideration is relevant to patient-based research, as the bulk of microarray analyses performed in this context involve multicellular samples.

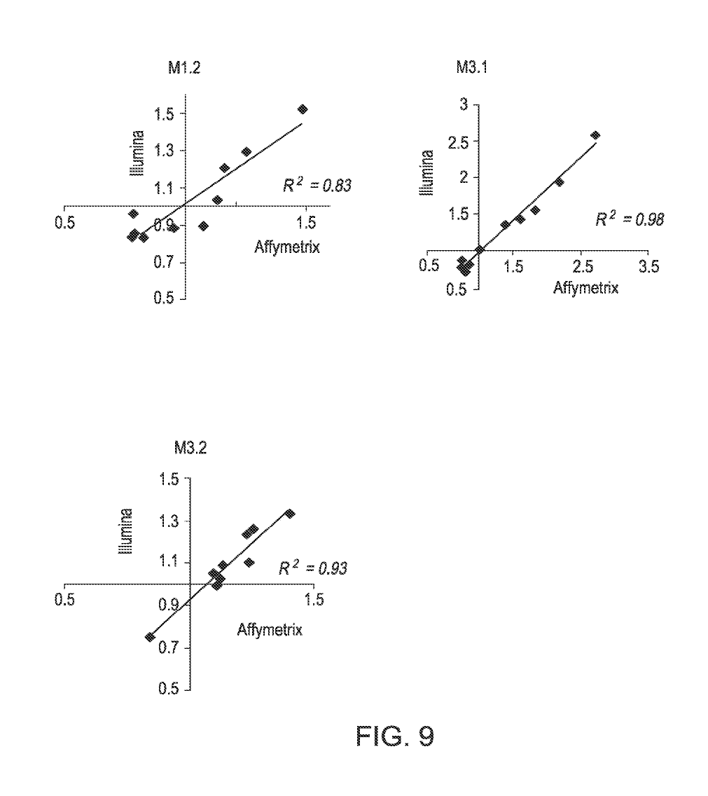

The modular expression data generated by Affymetrix and Illumina platforms were highly comparable (FIG. 9; transplant group Pearson correlation coefficient R²=0.83, 0.98 and 0.93, for M1.2, M3.1 and M3.2 respectively; p<0.0001). Taken together, these results demonstrate that modular transcriptional data can be reproduced across microarray platforms. This finding is of importance because it indicates that the “modular microarray scores” can be used to assess disease severity in patients derived independently of the microarray platform being used.

The module-level mining strategy described in this work may be used with a broad range of biological systems, and is particularly well suited for the analysis of other clinically relevant samples, such as tumors or solid organ biopsies.

Expression level vectors may be obtained from one or more of the modules and/or one or more of the genes provided in Table 3. Furthermore, depending on the disease expression profile and using the methods of the present invention it is possible to develop and further refine the modules and genes within the modules, as will be apparent to the skilled artisan based on the present invention. For example, depending on the level of specificity required, the number of data set, the number of patients, and the like, one or more new of different module that includes a different proportion of differentially expressed genes within the context of a given disease may be used to develop new modules based on the new data to form and organize arrays based on the new subset of transcripts, which define new vectors that represent an average expression level.

Tables 1, 2 and 3 are LENGTHY TABLES. The patent application contains a lengthy table section. A copy of the table is available in electronic form from the USPTO web site. An electronic copy of the table will also be available from the USPTO upon request and payment of the fee set forth in 37 CFR 1.19(b)(3), which is attached to this EFS filing and Tables 1, 2 and 3 are incorporated in their entirety by reference.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

In the claims, all transitional phrases such as “comprising,” “including, ” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### References

1. Golub, T. R. et al. Molecular classification of cancer: class discovery and class prediction by gene expression monitoring. Science 286, 531-7 (1999).
2. Alizadeh, A. A. et al. Distinct types of diffuse large B-cell lymphoma identified by gene expression profiling. Nature 403, 503-11 (2000).
3. Garber, K. Genomic medicine. Gene expression tests foretell breast cancer's future. Science 303, 1754-5 (2004).
4. van de Vijver, M. J. et al. A gene-expression signature as a predictor of survival in breast cancer. N Engl J Med 347, 1999-2009 (2002).
5. Pascual, V., Allantaz, F., Arce, E., Punaro, M. & Banchereau, J. Role of interleukin-1 (IL-1) in the pathogenesis of systemic onset juvenile idiopathic arthritis and clinical response to IL-1 blockade. J Exp Med 201, 1479-86 (2005).
6. Michiels, S., Koscielny, S. & Hill, C. Prediction of cancer outcome with microarrays: a multiple random validation strategy. Lancet 365, 488-92 (2005).
7. Ioannidis, J. P. Microarrays and molecular research: noise discovery? Lancet 365, 454-5 (2005).
8. Jarvinen, A. K. et al. Are data from different gene expression microarray platforms comparable? Genomics 83, 1164-8 (2004).
9. Tan, P. K. et al. Evaluation of gene expression measurements from commercial microarray platforms. Nucleic Acids Res 31, 5676-84 (2003).
10. Bammler, T. et al. Standardizing global gene expression analysis between laboratories and across platforms. Nat Methods 2, 351-6 (2005).
11. Irizarry, R. A. et al. Multiple-laboratory comparison of microarray platforms. Nat Methods 2, 345-50 (2005).
12. Larkin, J. E., Frank, B. C., Gavras, H., Sultana, R. & Quackenbush, J. Independence and reproducibility across microarray platforms. Nat Methods 2, 337-44 (2005).
13. Chaussabel, D. Biomedical literature mining: challenges and solutions in the ‘omics’ era. Am J Pharmacogenomics 4, 383-93 (2004).
14. Rhodes, D. R. et al. Mining for regulatory programs in the cancer transcriptome. Nat Genet 37, 579-83 (2005).
15. Segal, E., Friedman, N., Koller, D. & Regev, A. A module map showing conditional activity of expression modules in cancer. Nat Genet 36, 1090-8 (2004).
16. Mootha, V. K. et al. PGC-lalpha-responsive genes involved in oxidative phosphorylation are coordinately downregulated in human diabetes. Nat Genet 34, 267-73 (2003).
17. Segal, E., Friedman, N., Kaminski, N., Regev, A. & Koller, D. From signatures to models: understanding cancer using microarrays. Nat Genet 37 Suppl, S38-45 (2005).
18. Chaussabel, D. & Sher, A. Mining microarray expression data by literature profiling. Genome Biol 3, RESEARCH0055 (2002).
19. Bennett, L. et al. Interferon and granulopoiesis signatures in systemic lupus erythematosus blood. J Exp Med 197, 711-23 (2003).
20. Connolly, P. H. et al. Effects of exercise on gene expression in human peripheral blood mononuclear cells. J Appl Physiol 97, 1461-9 (2004).
21. Barrett, T. et al. NCBI GEO: mining millions of expression profiles--database and tools. Nucleic Acids Res 33, D562-6 (2005).
22. Ogawa, K., Oka, J., Yamakawa, J. & Higuchi, M. A single bout of exercise influences natural killer cells in elderly women, especially those who are habitually active. J Strength Cond Res 19, 45-50 (2005).
23. Woods, J. A., Evans, J. K., Wolters, B. W., Ceddia, M. A. & McAuley, E. Effects of maximal exercise on natural killer (NK) cell cytotoxicity and responsiveness to interferon-alpha in the young and old. J Gerontol A Biol Sci Med Sci 53, B430-7 (1998).
24. Tuma, R. S. Efforts aimed at reducing noise, data overload in microarrays. J Natl Cancer Inst 97, 1173-5 (2005).

1-51. (canceled)

52. A method for diagnosing systemic lupus erythematosus (SLE) comprising the steps of:
analyzing a sample from a patient suspected of having SLE based on one or more transcriptional modules that are indicative of SLE; and
determining whether the patient has SLE based on the presence, absence or level of expression of genes within one or more transcriptional modules.

53. The method of claim 52, wherein the transcriptional modules comprise 2, 3, 4, 5, 6, 7, 8, 9, or 10 genes, between 11 and 20 genes, or between 21 and 30 genes.

54. The method of claim 52, wherein the one or more transcriptional modules are selected from one or more of genes encoding for immunoglobulin chains and genes encoding interferon-inducible genes.

55. The method of claim 54, wherein the one or more genes encoding for immunoglobulin chains are selected from IGHM, IGJ, IGLL1, IGKC, IGHD and CD38.

56. The method of claim 54, wherein the one or more genes encoding interferon-inducible genes are selected from antiviral molecules, chemokines, and signaling molecules.

57. The method of claim 56, wherein the one or more antiviral molecules are selected from OAS1/2/3/L, GBP1, G1P2, ElF2AK2/PKR, MX1, and PML, the chemokines comprise CXCL10/IP-10, and the one or more signaling molecules are selected from STAT1, STAt2, IRF7, and ISGF3G.

58. The method of claim 52, wherein the level of expression of genes is determined using a probe array, PCR, quantitative PCR, bead-based assays, and combinations thereof.

59. The method of any of claims 58, further comprising using a computer algorithm to evaluate the measured levels of expression of one or more genes.

60. The method of claim 52, wherein the sample is whole blood, peripheral blood mononuclear cells, or sputum.

61. The method of claim 54, wherein the transcriptional modules is obtained by:
iteratively selecting gene expression values for one or more transcriptional modules by:
selecting for the module the genes from each cluster that match in every disease or condition;
removing the selected genes from the analysis; and
repeating the process of gene expression value selection for genes that cluster a sub-fraction of the diseases or conditions; and
iteratively repeating the generation of modules for each clusters until all gene clusters are exhausted.

62. The method of claim 61, wherein the clusters are selected from expression value clusters, keyword clusters, metabolic clusters, disease clusters, infection clusters, transplantation clusters, signaling clusters, transcriptional clusters, replication clusters, cell-cycle clusters, siRNA clusters, miRNA clusters, mitochondrial clusters, T cell clusters, B cell clusters, cytokine clusters, lymphokine clusters, heat shock clusters and combinations thereof,

63. A method for treating a patient for systemic lupus erythematosus (SLE) comprising the steps of:
analyzing a sample from a patient suspected of having SLE based on one or more transcriptional modules that are indicative of SLE; and
treating the patient if the presence, absence, or level of expression of genes within one or more transcriptional modules indicates that the patient has SLE.

64. A computer system comprising a relational database having records containing:
(a) information about one or more modules associated with systemic lupus erythematosus (SLE);
(b) information identifying genes associated with SLE; and
(c) a user interface allowing a user to selectively access the information contained in the records.
